(19) United States
(12) Patent Application Publication
(10) Pub. No.: US 2026/0124394 A1
(43) Pub. Date: May 7, 2026

(54) DRUG DELIVERY DEVICES, COMPONENTS FOR USE WITHIN DRUG DELIVERY DEVICES, AND METHODS OF OPERATING DRUG DELIVERY DEVICES

(71) Applicant: AMGEN INC., Thousand Oaks, CA (US)

(72) Inventors: Christopher Quon, Thousand Oaks, CA (US); Alireza Ashani, Woodland Hills, CA (US); Andrew Coles, Thousand Oaks, CA (US); Keng -Tong See, Camarillo, CA (US); Desheng Yin, Thousand Oaks, CA (US); Sergio Giordano, Varese (IT); Carlo Trabucchi, Monza Brianza (IT); Alberto Silvano, Milano (IT)

(21) Appl. No.: 18/704,334

(22) PCT Filed: Nov. 4, 2022
(86) PCT No.: PCT/US2022/048906

§ 371 Date: Apr. 24, 2024

Related U.S. Application Data
(60) Provisional application No. 63/276,384, filed on Nov. 5, 2021.

(51) Int. Cl. A61M 5/20

(52) U.S. Cl. CPC A61M 5/20; A61M 2005/2013; A61M 2005/206; A61M 2202/0007; A61M 2205/3553; A61M 2205/3584; A61M 2205/3592; A61M 2205/52

(57) ABSTRACT

Drug delivery devices, components for use within drug delivery devices, and methods of operating drug delivery devices are provided. A drug delivery device may include using a real-time clock of a wireless communication module to communicate data with a remote device when a main microcontroller is in a sleep mode. A drug delivery device may include disabling communication between a main microcontroller and a wireless communication module when the main microcontroller during an injection process. A drug delivery device may include determining an end of an injection process based on data from an insertion drive and an extrusion drive. A drug delivery device may include dual proximity sensors with unique thresholds. A drug delivery device may include electrostatic discharge protection and recovery.

### CROSS-REFERENCE TO RELATED APPLICATION

Priority is claimed to U.S. Provisional Patent Application No. 63/276,384, filed Nov. 5, 2021, the entire contents of which are hereby incorporated by reference herein.

### FIELD OF DISCLOSURE

The present disclosure generally relates to drug delivery devices. More specifically, the present disclosure relates to drug delivery devices, components for use within drug delivery devices, and methods of operating drug delivery devices having improved functionality.

### BACKGROUND

Numerous drug products are manufactured and packaged in, for example, a prefilled syringe (PFS) cartridge for use within a drug delivery device (e.g., an autoinjector (AI), a wearable drug delivery device, etc.). Associated drug delivery devices may include a host of electrically operated components (e.g., an insertion drive, an extrusion drive, a main microcontroller for controlling an injection process, a plurality of user interfaces, a wireless communication module for communication of drug delivery device configuration data and injection data with a remote device, etc.).

Drug delivery devices often use a real-time clock of a main microcontroller for synchronization of communication with a remote device. Thus, the main microcontroller may need to be operating in an active mode each time a remote device is communicatively connected to the drug delivery device.

Drug delivery devices often include a main microcontroller configured to control an injection process. Thus drug delivery device integrity and data security may be sacrificed during an injection process if a remote device can communicatively connect to the main microcontroller.

Drug delivery devices often determine an end of an injection process based on data from an extrusion drive status (e.g., a plunger rod is withdrawn from a syringe post medication injection, etc.). Thus, end of an injection process may be erroneously determined.

Drug delivery devices often use proximity sensors configured to assist a user to accurately place a proximal end of the drug delivery device next to an desired injection site. Multiple proximity sensors may produce erroneous results when common detection thresholds are used for each of the multiple sensors.

Drug delivery device often include a plurality of electrical components that produce electrostatic discharge (ESD) while a user administers an associated medication. Drug delivery devices may incur operational anomalies and/or damage due to the electrostatic discharge (ESD).

Drug delivery devices, components for use within drug delivery devices, and methods of operating drug delivery devices are needed that use a real-time clock of a wireless communication module to communicate data with a remote device when a main microcontroller is in a sleep mode. Drug delivery devices are needed that disable communication between a main microcontroller and a wireless communication module when the main microcontroller during an injection process. Drug delivery devices are needed that determine an end of an injection process based on data from an insertion drive and an extrusion drive. Drug delivery devices are needed that use dual proximity sensors with unique thresholds. Drug delivery devices are needed with electrostatic discharge protection and recovery.

### SUMMARY

A drug delivery device may include a housing configured to carry a syringe with a medication. The drug delivery device may also include an extrusion drive for selectively extruding the medication from the syringe during an injection process. The drug delivery device may further include a main microcontroller and a wireless communication module carried by the housing. The main microcontroller and the wireless communication module may be communicatively connected via a communication channel. The main microcontroller may include a first real-time clock. The main microcontroller may be configured to generate injection data based on the first real-time clock. The wireless communication module may include a periphery interface and a second real-time clock. The periphery interface may be configured to communicate the injection data to a remote device based on the second real-time clock.

In another embodiment, a method of operating a drug delivery device may include providing a main microcontroller communicatively connected with a wireless communication module via a communication channel. The main microcontroller may include a first real-time clock. The main microcontroller may be configured to generate injection data based on the first real-time clock and to control at least a portion of a medication injection process based on the injection data. The wireless communication module may include a periphery interface and a second real-time clock. The method may further include communicating injection data, via the periphery interface, based on the second real-time clock.

In a further embodiment, a non-transitory computer-readable medium storing computer-readable instructions that, when executed by one or more processors, may cause the one or more processors to receive a first real-time clock signal from a main microcontroller communicatively connected with a wireless communication module via a communication channel. The main microcontroller may be configured to generate injection data and to control at least a portion of a medication injection process. Execution of the instructions by the one or more processors may cause the one or more processors to further receive a second real-time clock signal from the wireless communication module. Execution of the instructions by the one or more processors may cause the one or more processors to further communicate the injection data via a periphery interface of the wireless communication module based on the second real-time clock.

In yet another embodiment, a drug delivery device may include a housing configured to carry a syringe with a medication. The drug delivery device may also include an extrusion drive for selectively extruding the medication from the syringe during an injection process. The drug delivery device may further include a main microcontroller communicatively connected with a wireless communication module via a communication channel. The main microcontroller may be configured to control at least a portion of a medication injection process. Communication via the serial communication channel may be disabled while the main microcontroller is controlling the at least the portion of the medication injection process.

In a further embodiment, a method of operating a drug delivery device may include controlling at least a portion of a medication injection process with a main microcontroller of the drug delivery device. The method may also include establishing a communication connection between the main microcontroller and a wireless communication module of the drug delivery device. The method may further include disabling communication across the communication connection while the main microcontroller is controlling at least the portion of the medication injection process.

In another embodiment, a non-transitory computer-readable medium storing computer-readable instructions that, when executed by one or more processors, may cause the one or more processors to communicatively connect a main microcontroller with a wireless communication module via a communication channel, wherein the main microcontroller is configured to control at least a portion of a medication injection process. Further execution of the instructions by the one or more processors may cause the one or more processors to further disable communication via the communication channel while the main microcontroller is controlling the at least the portion of the medication injection process.

In a yet another embodiment, a drug delivery device may include a housing configured to carry a syringe with a medication for extrusion during an injection process. The drug delivery device may also include an insertion drive system (IDS) configured to insert a needle of the syringe into a patient before extrusion of the medication during the injection process and retract the needle into the housing after extrusion of the medication. The drug delivery device may further include an extrusion drive system (EDS) comprising a plunger rod configured to move through the syringe to extrude the medication out of the needle during the injection process. The drug delivery device may yet further include a microcontroller configured to determine end of the injection process drug delivery device based on the completion of movement of the plunger rod of the EDS through the syringe during the injection process.

In yet another embodiment, a method of operating a drug delivery device may include driving an insertion drive system (IDS) forward to insert the syringe needle. The method may also include driving an extrusion drive system (EDS) forward the plunger rod to extrude the fluid. The method may further include determining end of an injection in a drug delivery device based on the completion of EDS movement when driving forward the plunger rod to extrude the fluid.

In yet a further embodiment, a non-transitory computer-readable medium storing computer-readable instructions that, when executed by one or more processors, may cause the one or more processors to drive an insertion drive system (IDS) forward to insert the syringe needle. Execution of the instructions by the one or more processors may cause the one or more processors to further drive an extrusion drive system (EDS) forward the plunger rod to extrude the fluid. Execution of the instructions by the one or more processors may cause the one or more processors to further determine end of an injection in a drug delivery device based on the completion of EDS movement when driving forward the plunger rod to extrude the fluid.

In another embodiment, a drug delivery device may include a housing configured to carry a syringe with a medication. The drug delivery device may also include an extrusion drive for selectively extruding the medication from the syringe during an injection process. The drug delivery device may further include a first capacitance sensor to generate a first output. The drug delivery device may yet further include a second capacitance sensor to generate a second output. The drug delivery device may also include a microcontroller that may be configured to enable an injection process based on a comparison of the first output with a first threshold and comparison of the second output with a second threshold. The first threshold may be different than the second threshold.

In a further embodiment, a method of operating a drug delivery device may include generating a first capacitance sensor output with a first capacitance sensor carried by a housing of a drug delivery device. The method may also include generating a second capacitance sensor output with a second capacitance sensor carried by the housing of the drug delivery device. The method may further include enabling an injection process based on a comparison of the first output with a first threshold and comparison of the second output with a second threshold. The first threshold may be different than the second threshold.

In yet another embodiment, a non-transitory computer-readable medium storing computer-readable instructions that, when executed by one or more processors, may cause the one or more processors to generate a first capacitance sensor output. Execution of the instructions by the one or more processors may cause the one or more processors to further generate a second capacitance sensor output. Execution of the instructions by the one or more processors may cause the one or more processors to further enable an injection process based on a comparison of the first output with a first threshold and comparison of the second output with a second threshold. The first threshold may be different than the second threshold.

In yet a another embodiment, a drug delivery device may include a housing configured to carry a syringe with a medication. The drug delivery device may also include an extrusion drive for selectively extruding the medication from the syringe during an injection process. The drug delivery device may further include a plurality of electronic components. The drug delivery device may yet further include at least one electrostatic discharge (ESD) protection device including a watchdog circuit and an ESD recovery module.

In yet a further embodiment, a method of operating a drug delivery device may include providing at least one drive mechanism. The method may also include providing a plurality of electronic components. The method may further include providing at least one electrostatic discharge protection device including a watchdog circuit and a recovery module.

In another embodiment, a non-transitory computer-readable medium storing computer-readable instructions that, when executed by one or more processors, may cause the one or more processors to provide at least one electrostatic discharge (ESD) protection device including a watchdog circuit and a recovery module to provide ESD protection for at least one drive mechanism and a plurality of electronic components.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicated of the presence or absence of particular elements in any of the exemplary embodiments, except as may be explicitly delineated in the corresponding written description. Also, none of the drawings are necessarily to scale.

FIGS. 1A-D depict an example drug delivery system;

FIGS. 2A-G depict various views of an example drug delivery device;

FIGS. 3A-E depict block diagrams for an example drug delivery system and example methods of operating a drug delivery device;

FIGS. 4A and 4B depict example real-time clocks for data communication within a drug delivery device;

FIG. 5 depicts an example data communication disabling device for use within a drug delivery device;

FIGS. 6A-N and P-R depict an example proximity sensor for use within a drug delivery device;

FIGS. 7A-G depict example thresholds for use with a proximity sensor of a drug delivery device;

FIGS. 8A-N and P-Z depict example thresholds for use with a proximity sensor of a drug delivery device; and

FIGS. 9A-E depict example electrostatic discharge protection for use within a drug delivery device.

### DETAILED DESCRIPTION

Drug delivery devices, components for use within drug delivery devices, and methods of operating drug delivery devices are provided. As described herein, a drug delivery device may include using a real-time clock of a wireless communication module to communicate data with a remote device when a main microcontroller is in a sleep mode. Thereby, the main microcontroller may remain in a sleep mode during periods of time that the drug delivery device is synchronized with a remote device. Power consumption of the main microcontroller may be lower when the microcontroller is in a sleep mode compared to when the microcontroller is operating in an active mode.

As also described herein, a drug delivery device may include disabling communication between a main microcontroller and a wireless communication module when the main microcontroller during an injection process. Thereby, main microcontroller processing resources may be devoted to control of the injection process. Similarly, security of the drug delivery device may increase during the injection process.

As further described herein, a drug delivery device may include determining an end of an injection process based on data from an insertion drive and/or an extrusion drive. For example, the drug delivery device may be configured to deliver a particular medication as a single dose or sequentially deliver the medication in a series of individual doses over a period of time. When the medication is delivered sequentially in a series of individual doses, an end of any given portion of the associated injection process (i.e., each individual dose) may be, for example, based on extrusion drive data. When the medication is delivered in a single dose, an end of the associated injection process may be, for example, based on both extrusion drive data and insertion drive data.

As yet further described herein, a drug delivery device may include dual proximity sensors (e.g., capacitance sensors, etc.) configured to, for example, detect when a proximal end of the drug delivery device is proximate a desired injection site (e.g., a skin surface of a user, etc.). Each sensor may be associated with unique thresholds (e.g., an “in contact” threshold, an “out of contact” threshold, etc.). A main microcontroller may determine proximity based on two different thresholds for each of two sensors (four thresholds total) configured to impart hysteresis for each sensor output.

As also described herein, a drug delivery device may include electrostatic discharge (ESD) protection and recovery. For example, the drug delivery device may include at least one ESD watchdog circuit configured to detect an ESD event. The drug delivery device may also include an ESD recovery module configured to attempt drug delivery device operation recovery based on an output of an ESD watchdog circuit.

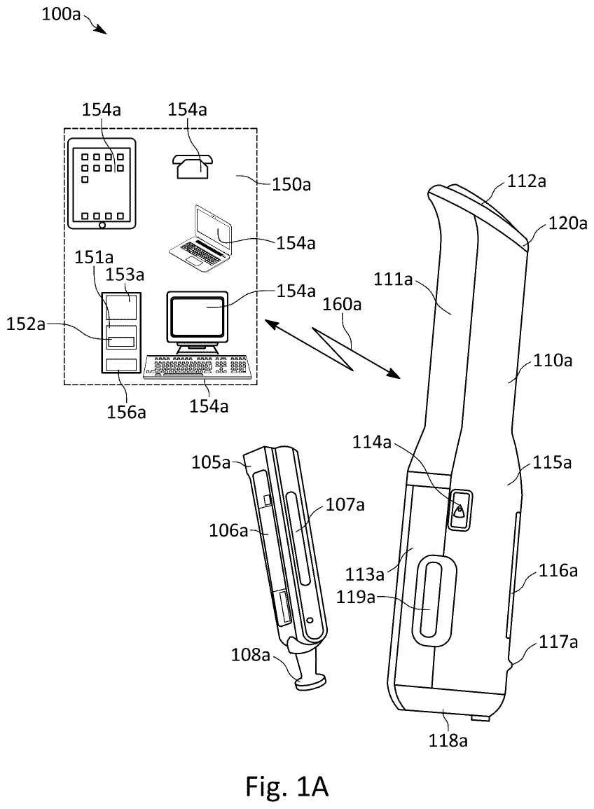

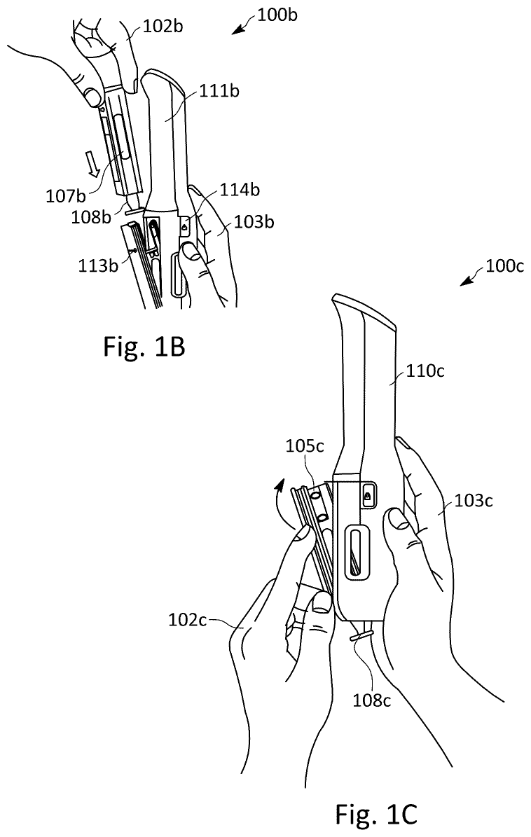

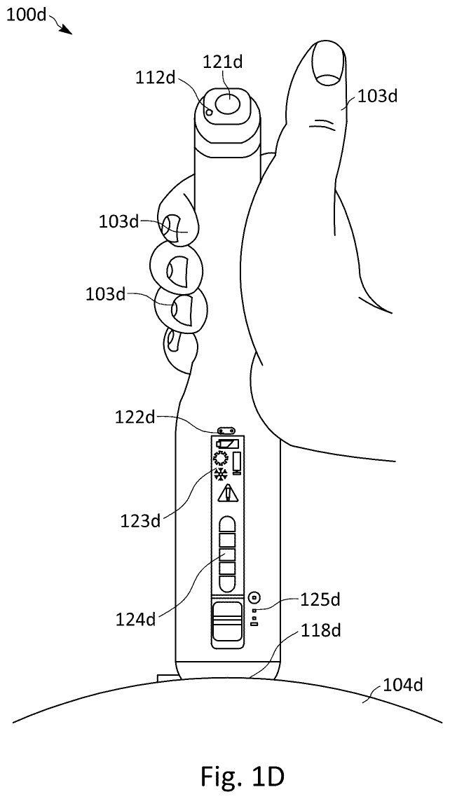

Turning to FIGS. 1A-D, a drug delivery system 100a-d may include a drug delivery device 110a, c with associated medication cartridge 105a-c. While only one drug delivery device 110a,c and one medication cartridge 105a-c are included for illustrative purposes, a drug delivery system 100a-d may include any number of drug delivery devices 110a,c and/or medication cartridges 105a-c.

The medication cartridge 105a-c may include an information label 106a (e.g., print, a near-field communication device, a bar code, a QR code, etc.), a prefilled syringe 107a, and a needle cap 108a-c. The drug delivery device 110a,c may include a handle 111a,b configured to be gripped by a hand 103a-d of a user with a thumb of the user proximate a distal end 120a. The drug delivery device 110a,c may include an injection initiation button 112a,d, for example, proximate the distal end 120a.

The drug delivery device 110a,c may include a cartridge receptacle 113a,b and a cartridge receptacle open device 114a,b. A user may activate the cartridge receptacle open device 114a,b to open the cartridge receptacle 113a,b and insert the medication cartridge 105a-c, as illustrated in FIGS. 1B and 1C. Once the medication cartridge 105a-c is within the drug delivery device 110a,c, the prefilled syringe 107a may be visible through a viewing window 119a.

The drug delivery device 110a,c may include a housing portion 115a, a status indicator 121d, a speaker 122d, an error display 123d, an injection progress indicator, and an injection speed switch 125d. Once a user selects an injection speed via switch 125d, the user may place the proximal end 118d of the drug delivery device 110a,c next to the injection site 104d, and press the injection initiation button 112d to initiate an injection.

The drug delivery system 100a-d may also include at least one remote site 150a. While only one remote site 150a is included in FIG. 1A for illustrative purposes, a drug delivery system 100a-d may include any number of remote sites 150a. Any given remote site may include at least one non-transitory computer-readable medium 151a having a module 152a, and at least one processor 153a. The module 152a may include computer-readable instructions that, when executed by the at least one processor 153a, may cause the processor 153a to communicate drug delivery device configuration data and/or injection data between the drug delivery device 110a,c and the remote site 150a. As described in more detail elsewhere herein, drug delivery device configuration data may be representative of, for example, medication cartridge configuration data (e.g., manually entered via user interface, automatically retrieved via cartridge QR code, automatically retrieved via cartridge bar code, automatically received from cartridge manufacture, etc.), real-time clock configuration data, communication link configuration data, end of injection detection configuration data, proximity sensor threshold configuration data, electrostatic discharge (ESD) protection configuration data, etc. Injection data may be representative of, for example, a medication, a medication cartridge, a day of an injection, a time of an injection, an injection speed, a drug delivery device proximity, a drug delivery device tilt, a drug delivery device data request from a remote device, a drug delivery device data transmission to a remote device, an end of delivery, detection of an ESD event, etc. Drug delivery device data may be representative of, for example, a sub-combination or a combination of drug delivery device configuration data with injection data.

As illustrated in FIG. 1A, a drug delivery device 110a,c may be communicatively coupled to a network interface 156a via a network 160a to communicate, for example, drug delivery device configuration data and/or injection data between the drug delivery device 110a,c and a remote site 150a. A non-transitory computer-readable medium 151a having a module 152a may be, as examples, embodied in either firmware or computer-readable code.

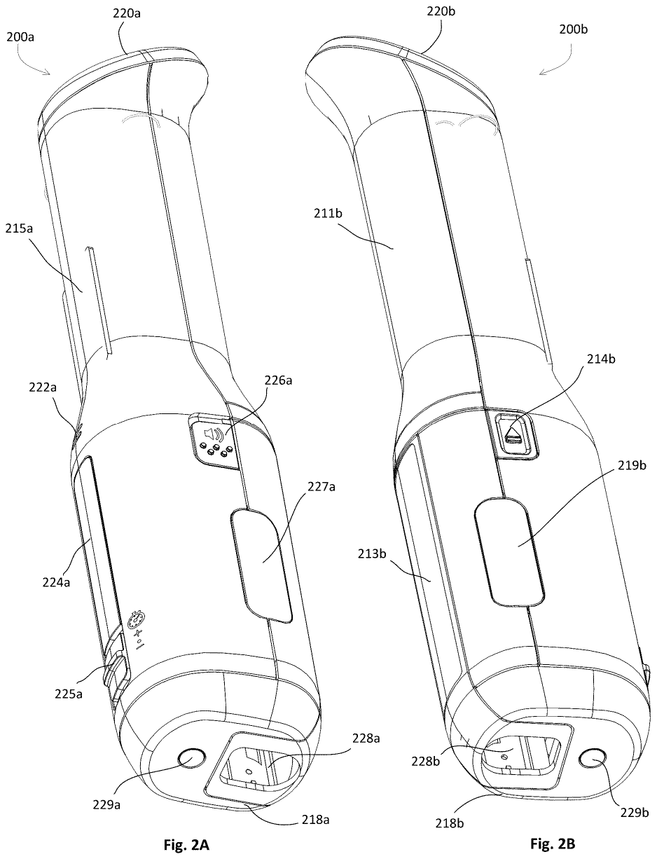

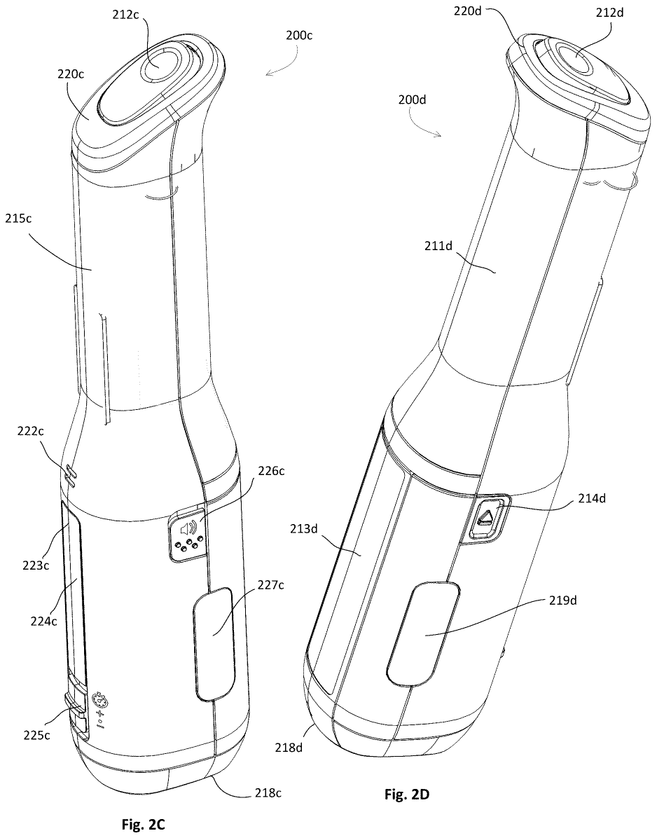

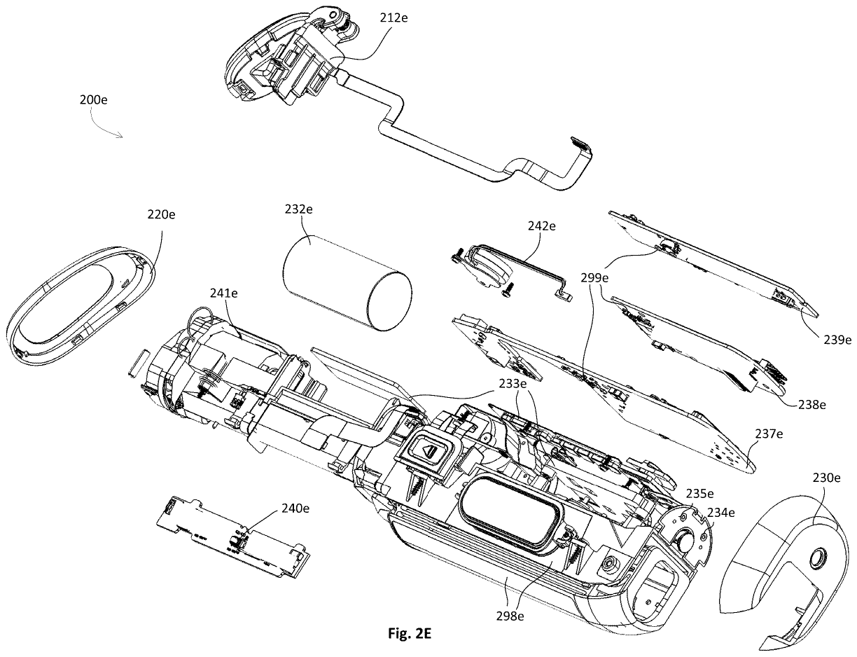

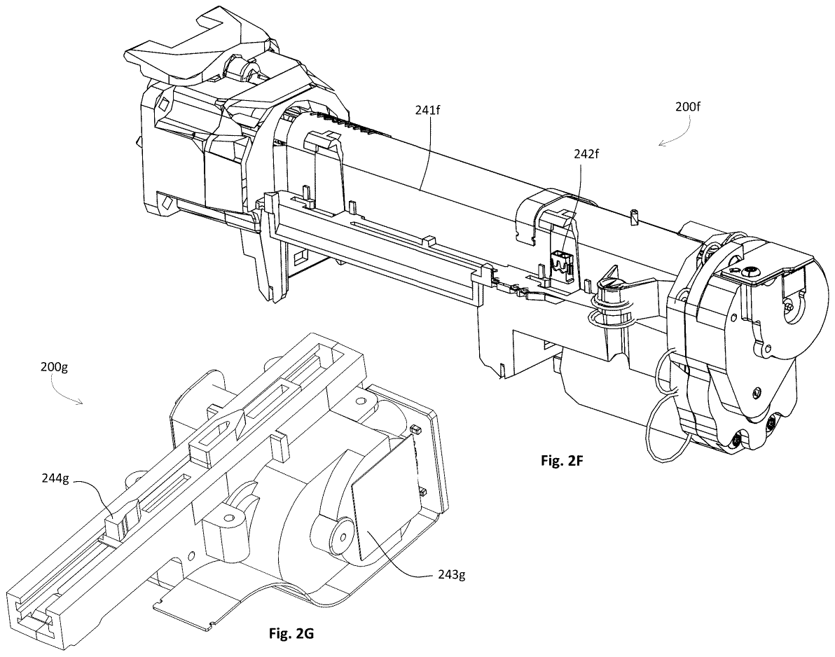

With reference to FIGS. 2A-G, a drug delivery device 200a-g may include a handle 211b,d. The drug delivery device 200a-g may also include a housing portion 215a,c. The drug delivery device 200a-g may also include a speaker 222a,c. The drug delivery device 200a-g may also include an information display 224a,c. The drug delivery device 200a-g may also include a proximal end 218a,b with proximity sensor 229a,b and needle/needle cap aperture 228a,b. The drug delivery device 200a-g may also include a distal end 220a-d with injection initiation button 212c,d. The drug delivery device 200a-g may also include an injection speed selector 225a,c.

The drug delivery device 200a-g may also include a medication cartridge receptacle 213b,d. The drug delivery device 200a-g may also include a medication cartridge receptacle open switch 214b,d. The drug delivery device 200a-g may also include a sound on/off switch 226a,c. The drug delivery device 200a-g may also include a first viewing window 227a,c. The drug delivery device 200a-g may also include a second viewing window 219b,c. The drug delivery device 200a-g may also include a battery 232e. The drug delivery device 200a-g may also include an insertion drive 243g having an insertion drive orientation sensor 244g. The drug delivery device 200a-g may also include an extrusion drive 241e,f having an extrusion drive orientation sensor 242f. The drug delivery device 200a-g may also include a distal end cap 220e. The drug delivery device 200a-g may also include a proximal end cap 230e.

The drug delivery device 200a-g may also include a medication cartridge receptacle 213b,d. The drug delivery device 200a-g may also include electrostatic discharge (ESD) protection having, for example, at least one mechanical solution (e.g., make device conductive, design Insulation/ESD shield 233e and/or distance between enclosure and electronic component 298e) and at least one electronic solution (e.g., at least one ESD protective component/circuit 299e on hardware, at least one zener diode circuit 999f).

The drug delivery device 200a-g may also include an injection initiation/status assembly 212e. The drug delivery device 200a-g may also include a cartridge eject button assembly 114e. The drug delivery device 200a-g may also include a first proximity sensor 234e (e.g., a capacitive sensor, etc.). The drug delivery device 200a-g may also include a second proximity sensor 235e (e.g., a capacitive sensor, etc.). The drug delivery device 200a-g may also include a proximity sensor attachment. The drug delivery device 200a-g may also include a main lower printed circuit board 237e. The drug delivery device 200a-g may also include a main upper printed circuit board 238e. The drug delivery device 200a-g may also include a progress bar printed circuit board 239e. The drug delivery device 200a-g may also include a handle printed circuit board 240e.

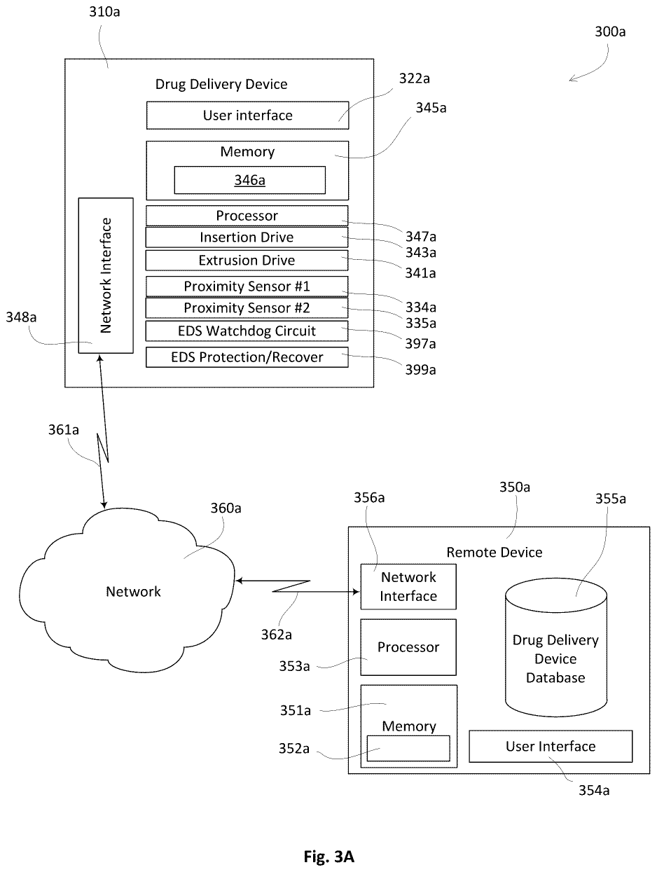

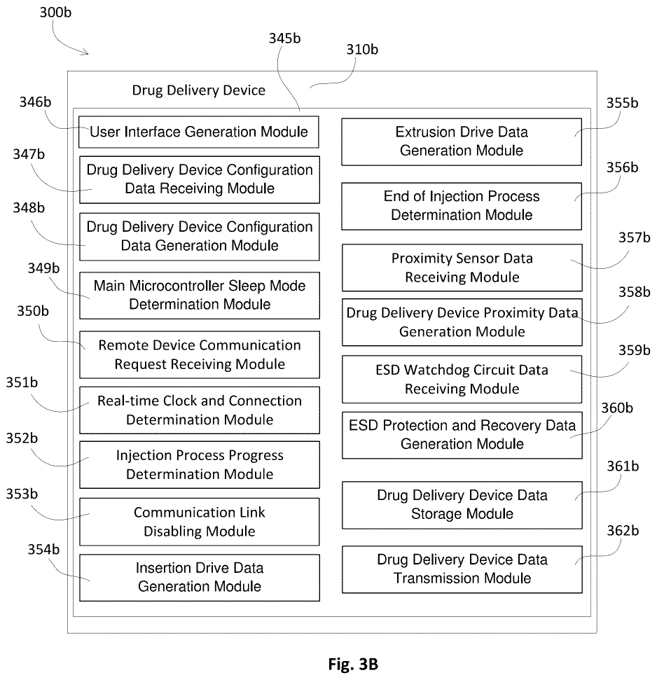

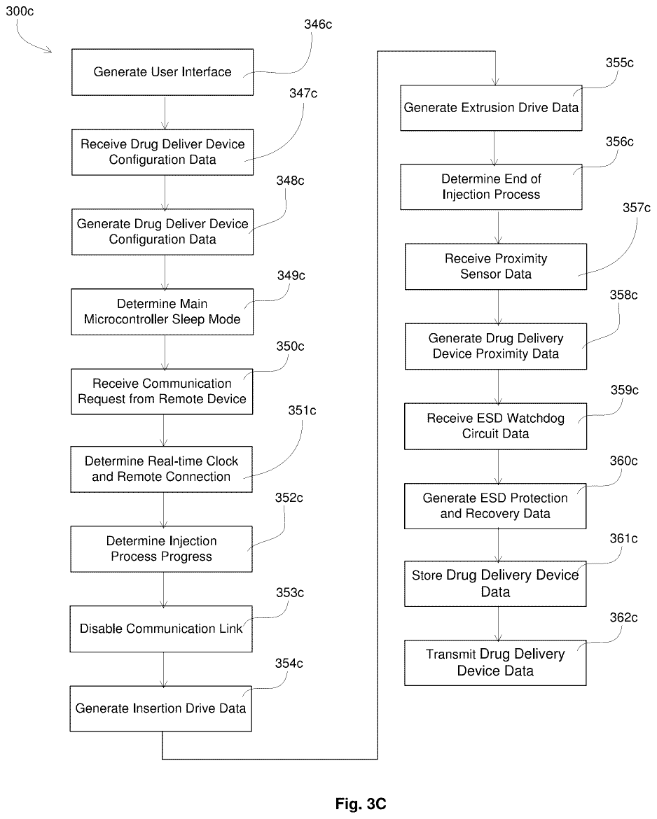

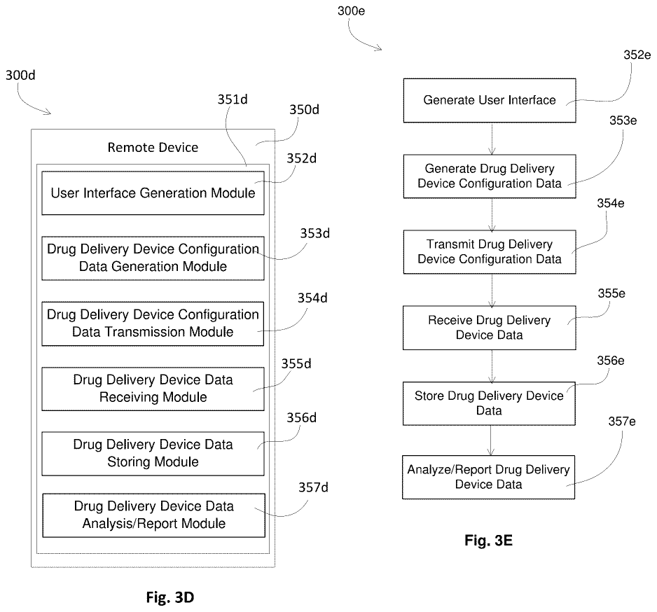

Turning to FIGS. 3A-E, a drug delivery system 300a-e may include a drug delivery device 310a-c in communication with a remote device (e.g., a server) 350a,d,e via a network 360a. The drug delivery device 310a-c may be similar to, for example, the drug delivery device 110a,c of FIGS. 1A and 1B, respectively, or the drug delivery device of FIGS. 2A-N. The remote device 350a,d,e may be similar to, for example, the remote site 150a of FIG. 1A.

The drug delivery system 300a-e may implement communications between the drug delivery device 310a-c and the remote device 350a,d,e (e.g., a remote server, cloud-based resources, etc.) to provide, for example, drug delivery device configuration data and/or injection data to a drug delivery device database 355a.

For clarity, only one drug delivery device 310a-c is depicted in FIG. 3A. While FIG. 3A depicts only one drug delivery device 310a-c, it should be understood that any number of drug delivery device 310a-c may be supported. A drug delivery device 310a-c may include a memory 345a and a processor 347a for storing and executing, respectively, a module 346a. The module 346a stored in the memory 345a as a set of computer-readable instructions, may be related to an application for configuration of a drug delivery device, automatically control of an injection process, and generation of injection data.

As described in detail herein, the module 346a may facilitate interaction between an associated drug delivery device 310a-c and a remote device 350a,d,e. For example, the processor 347a, further executing the module 346a, may facilitate communications between a remote device 350a,d,e and a drug delivery device 310a-c via a network interface 348a, a communication link 361a, a network 360a, a remote device communication link 362a, and a remote device network interface 356a.

The drug delivery device 310a-c may include an insertion drive 343a, an extrusion drive 341a, a first proximity sensor 334a, a second proximity sensor 335a, an electrostatic discharge (ESD) watchdog circuit 397a, and ESD protection/recovery 399a. A drug delivery device 310a-c may include a user interface 322a which may be any type of electronic display device, such as touch screen display, a liquid crystal display (LCD), a light emitting diode (LED) display, a plasma display, a cathode ray tube (CRT) display, or any other type of known or suitable electronic display along with a user input device. A user interface 322a may exhibit a user interface (e.g., any user interface 121d, 123d, 124d, 154a, etc.) which depicts a user interface for configuring a drug delivery device 310a-c to communicate with a remote device 350a,d,e.

The network interface 360a may be configured to facilitate communications between a drug delivery device 310a-c and a remote device 350a,d,e via any wireless communication network 360a, including for example a Bluetooth low energy (BLE) device, a wireless LAN, MAN or WAN, WiFi, the Internet, or any combination thereof. Moreover, a drug delivery device 310a-c may be communicatively connected to a remote device 350a,d,e via any suitable communication system, such as via any publicly available or privately owned communication network, including those that use wireless communication structures, such as wireless communication networks, including for example, wireless LANs and WANs, satellite and cellular telephone communication systems, etc. A drug delivery device 310a-c may cause, for example, drug delivery device configuration data and/or injection data to be transmitted to, and stored in, for example, a remote device 350a,d,e, memory 351a, and/or a drug delivery device database 355a.

A remote device 350a,d,e may include a user interface 354a, a memory 351a, and a processor 353a for storing and executing, respectively, a module 352a. The module 352a, stored in the memory 351a as a set of computer-readable instructions, may facilitate applications related to controlling a drug delivery injection process. The module 352a may also facilitate communications between the remote device 350a,d,e and a drug delivery device 310a-c via a network interface 356a, and the network 360a, and other functions and instructions.

A remote device 350a,d,e may be communicatively coupled to a drug delivery device 310a-c. While the drug delivery device database 355a is shown in FIG. 3A as being communicatively coupled to the remote device 350a, it should be understood that the drug delivery device database 355a may be located within separate remote servers (or any other suitable computing devices) communicatively coupled to the remote device 350a,d,e. Optionally, portions of drug delivery device database 355a may be associated with memory modules that are separate from one another, such as a memory 345a of a drug delivery device 310a-c.

A drug delivery device 310a-c may include a user interface generation module 346b, a drug delivery device configuration data receiving module 347b, a drug delivery device configuration data generation module 348b, a main microcontroller sleep mode determination module 349b, a remote device communication request receiving module 350b, a real-time clock and connection determination module 351b, an injection process progress determination module 352b, a communication link disabling module 353b, an insertion drive data generation module 354b, an extrusion drive data generation module 355b, an end of injection process determination module 356b, a proximity sensor data receiving module 357b, a drug delivery device proximity data generation module 358b, an electrostatic discharge (ESD) watchdog circuit data receiving module 359b, an ESD protection and recovery data generation module 360b, a drug delivery device data storage module 361b, and a drug delivery device data transmission module 362b, for example, stored on a memory 345b as a set of computer-readable instructions. In any event, the modules 346b-362b may be similar to, for example, the module 346a of FIG. 3A.

A method of operating a drug delivery device 310c may be implemented by a first processor (e.g., processor 347a) executing, for example, at least a portion of modules 346b-362b. In particular, processor 347a may execute the user interface generation module 346b to cause the processor 347a to, for example, generate a user interface 375 (block 346c). The user interface may allow a user to enter and/or view, for example, drug delivery device configuration data and/or injection data.

Processor 347a may execute the drug delivery device configuration data receiving module 347b to cause the processor 347a to, for example, receive drug delivery device configuration data from a remote device, etc. (block 347c). Processor 347a may execute the drug delivery device configuration data generation module 348b to cause the processor 347a to, for example, generate drug delivery device configuration data (block 348c). Drug delivery device configuration data may be representative of, for example, medication cartridge configuration data (e.g., manually entered via user interface, automatically retrieved via cartridge QR code, automatically retrieved via cartridge bar code, automatically received from cartridge manufacture, etc.), real-time clock configuration data, communication link configuration data, end of injection detection configuration data, proximity sensor threshold configuration data, electrostatic discharge (ESD) protection configuration data, etc.

Processor 347a may execute the main microcontroller sleep mode determination module 349b to cause the processor 347a to, for example, determine whether the main microcontroller is currently in a sleep mode or an active mode (block 349c). For example, the processor 347a may determine whether the main microcontroller is currently in a sleep mode or an active mode (block 349c) based on data provided by the main microcontroller.

Processor 347a may execute the remote device communication request receiving module 350b to cause the processor 347a to, for example, receive a request from a remote device (block 350c). Processor 347a may execute the real-time clock and connection determination module 351b to cause the processor 347a to, for example, determine a real-time clock (e.g., a real-time clock of a main microcontroller, a real-time clock of a wireless communication module, etc.) to connect to the remote device (block 351c).

Processor 347a may execute the injection process progress determination module 352b to cause the processor 347a to, for example, determine a progress of a current injection process (block 352c). Processor 347a may execute the communication link disabling module 353b to cause the processor 347a to, for example, disable a communication link 569 (block 353c). Processor 347a may execute the insertion drive data generation module 354b to cause the processor 347a to, for example, control and/or monitor an insertion drive (block 354c). Processor 347a may execute the extrusion drive data generation module 355b to cause the processor 347a to, for example, control and/or monitor an extrusion drive (block 355c).

Processor 347a may execute the end of injection process determination module 356b to cause the processor 347a to, for example, determine an end of an injection process (block 356c). For example, the processor 347a may determine an end of an injection process based on insertion drive data, extrusion drive data, a combination of the insertion drive data and the extrusion drive data, etc. For electromechanical autoinjectors, determining end of injection for autoinjectors may ensure safe and effective injections. End of injection determination relies on the use of algorithm programmed in software to assess the signals and data from associated hardware throughout the injection process.

An autoinjector may include software, printed circuit board assemblies (PCBAs), an Extrusion Drive System (EDS) with plunger rod for fluid extrusion, and an Insertion Drive System (IDS) for needle insertion and retraction. The following provides typical injection process after a separate cartridge with prefilled syringe is inserted into the autoinjector and injection is initiated by button press: 1) IDS may drive forward to insert the syringe needle; 2) EDS may drive forward the plunger rod to extrude the fluid; 3) EDS may partially retract plunger rod; and 4) IDS may retract the syringe needle

Software logic can be used to determine end of injection after the IDS retracts the syringe needle. Additional software logic/algorithm can be added to determine end of injection based on the completion of EDS movement when driving forward the plunger rod to extrude the fluid. By adding additional software logic, the autoinjector enables a more accurate determination for end of injection in comparison to determining end of injection after the IDS retracts the syringe needle.

Processor 347a may execute the proximity sensor data receiving module 357b to cause the processor 347a to, for example, receive proximity sensor data (block 357c). Processor 347a may execute the drug delivery device proximity data generation module 358b to cause the processor 347a to, for example, generate drug delivery device proximity data (block 358c). Processor 347a may execute the ESD watchdog circuit data receiving module 359b to cause the processor 347a to, for example, receive ESD watchdog data (block 359c). Processor 347a may execute the ESD protection and recovery data generation module 360b to cause the processor 347a to, for example, generate ESD protection and recovery data (block 360c). Processor 347a may execute the drug delivery device data storage module 361b to cause the processor 347a to, for example, store drug delivery device configuration data and/or injection data (block 361c). Processor 347a may execute the drug delivery device data transmission module 362b to cause the processor 347a to, for example, transmit drug delivery device configuration data and/or injection data (block 362c).

A remote device 350a,d may include a user interface generation module 352d, a drug delivery device configuration data generation module 353d, a drug delivery device configuration data transmission module 354d, a drug delivery device data receiving module 355d, a drug delivery device data storing module356d, and a drug delivery device data analysis/report module 457d for example, stored on a memory 351d as a set of computer-readable instructions. In any event, the modules 352d-357d may be similar to, for example, the module 352a of FIG. 3A.

A method of operating a remote device 300e may be implemented by a processor (e.g., processor 353a) executing, for example, at least a portion of modules 352d-357d. In particular, processor 353a may execute the user interface generation module 352d to cause the processor 353a to, for example, generate a user interface 375, etc. (block 352e).

Processor 353a may execute the drug delivery device configuration data generation module 353d to cause the processor 353a to, for example, generate drug delivery device configuration data (block 353e). Processor 353a may execute the drug delivery device configuration data transmission module 354d to cause the processor 353a to, for example, transmit drug delivery device configuration data (block 354e). Drug delivery device configuration data may be representative of, for example, medication cartridge configuration data (e.g., manually entered via user interface, automatically retrieved via cartridge QR code, automatically retrieved via cartridge bar code, automatically received from cartridge manufacture, etc.), real-time clock configuration data, communication link configuration data, end of injection detection configuration data, proximity sensor threshold configuration data, electrostatic discharge (ESD) protection configuration data, etc.

Processor 353a may execute the drug delivery device data receiving module 355d to cause the processor 353a to, for example, receive drug delivery device data (block 355e). Processor 353a may execute the drug delivery device data storing module 356d to cause the processor 353a to, for example, store drug delivery device data (block 356e). Processor 353a may execute the drug delivery device data analysis/report module 357d to cause the processor 353a to, for example, analyze and report drug delivery device data (block 357e).

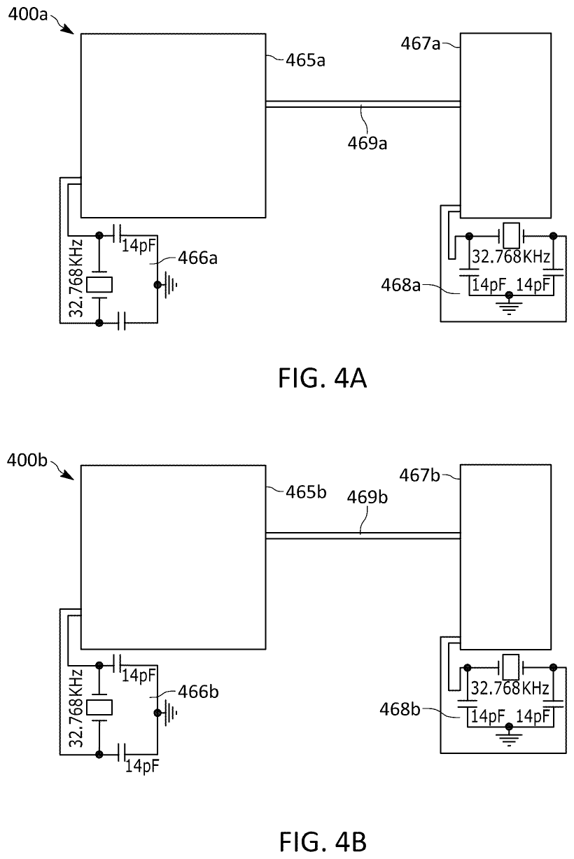

With reference to FIGS. 4A and 4B, a drug delivery device 400a,b may include main microcontroller 465a,b having a first real-time clock 466a,b communicatively connected to a wireless communication module 467a,b having a second real-time clock 468a,b via a communication channel 469a,b.

Real time clock (RTC) is one of the main components of any embedded devices, specifically medical devices, which is used for keeping track of time and date. As the autoinjector device is used for timely delivery of medications, it is important to have a correct time of the device. Device time is set to UTC and with minimum drift over time to maintain an accurate timing. This disclosure relates to the real time clock utilization of the autoinjector device (ATC). The autoinjector consists of two embedded components, main microcontroller (uC) and the BLE module. The main uC has essential peripheral accessories on its chip, referred to as System On a Chip (SOC). Real time clock is one of the embedded sub-components of this SOC. It has a crystal oscillator with the crystal frequency of 32.768 KHZ which would run during the active cycle of the main uC to provide a live real time. Additionally, the BLE module which handles the Bluetooth connectivity functions and has less power consumption also has its own RTC. The two components, main uC and BLE module, communicate via a serial communication channel (UART). To have any data exchange between the two, they both must be running in active mode. An external BLE capable device can connect and pair to the device and read the device time for synchronization purposes.

In the event of a time read request from an external BLE capable device, if for any reason, the autoinjector, and consequently the main uC go into sleep mode, the UART communication between main uC and BLE module would no longer be active. As a result, even though the RTC on the main uC would still be ticking, the live clock time would not be reported to the BLE module, and external BLE capable device. To resolve this issue, the RTC clock on the BLE module was utilized as an alternative to main uC RTC. The BLE module uses significantly lower power, compared to main uC, so it can stay on in active mode for an extended period of time, while the main uC and communication channel between main and BLE can be in sleep mode/inactive. This approach ensures a live real time whenever requested by an external BLE capable device.

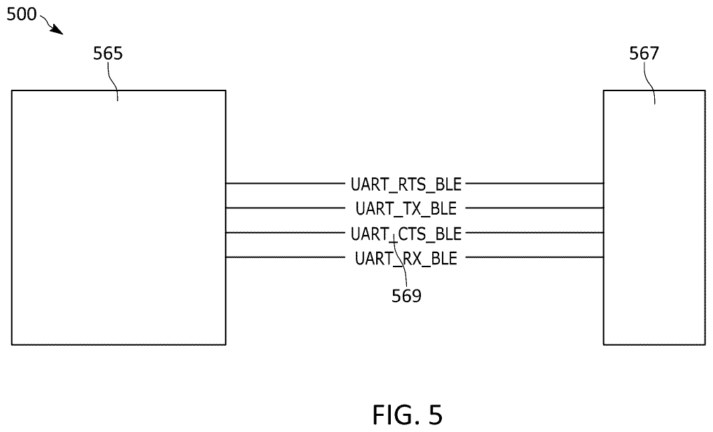

Turning to FIG. 5, a drug delivery device 500 may include main microcontroller 565 communicatively connected to a wireless communication module 567 via a communication channel 569.

Medical devices can potentially be consisted of more than one processing modules that are interconnected and communicate data between them. If any of these modules can connect/communicate to the outside boundaries of the system, these interconnections are open channels that can potentially introduce a security hole in the system. As an example of such systems, autoinjector is a sensitive device that is used for delivering the drug dosage to the patient. it is vital to ensure that during the dose administration, the external communication channels be disabled to ensure that no middleman could potentially interfere with the dose administration critical functions.

In the autoinjector design architecture, there may be two micro-controllers that each handle certain processes, one is the main micro-controller, which is from STM32 family, and the second micro-controller is in the BLE module, which is from nRF52 family. Main micro-controller handles all the critical functions of the autoinjector related to drug administration, such as drug extrusion, needle insertion, and needle retraction. It also serves all other non-critical functions of the autoinjector. BLE micro-controller handles the Bluetooth connectivity functions and has less power consumption, relative to the main micro-controller. They can communicate with each other via different communication methods, such as I2C, GPIOs, SPI, or UART. In this specific design, UART is utilized for communication between the two. The communication protocol between the two micro controllers is based on a two-way asynchronous communication with flow controls.

The BLE module is an open port for interfacing with another BLE capable device such as a mobile device, and as it is connected to the main micro-controller via UART, it can send and receive data to the main micro-controller and occupy main micro-controller's processing time. Since the BLE module is the only non-physical communication port to the device, it is susceptible to cybersecurity attacks by the man-in-the-middle and Unauthorized Direct Data Access (UDDA). In a cybersecurity attack scenario, the BLE micro-controller can be accessed by the man-in-the-middle and it can flood the main micro-controller with unwanted requests, potentially occupying main micro-controller's processing bandwidth. Even though the main micro-controller handles the processes based on interrupt priorities, such attack could potentially impact its functionality. During the injection process, it is essential to prevent any interruption to the main micro-controller to reduce the risk of device malfunction and keep the main micro-controller focused on processing the injection's critical functions. To prevent the cybersecurity attack scenario during the injection, and to ensure proper handling of the injection processes by the main micro-controller, the improvement was introduced to disable any communication method between the two micro-controllers, in this case, the UART communication channel between the main micro-controller and the BLE module. The main micro-controller will still process the injection tasks, so there is no impact to the overall injection process. It significantly improves the cybersecurity of the device during the injection and eliminate any risk of giving control of main micro-controller to man-in-the-middle as the access port will be disabled, hence ensuring an interrupt free injection.

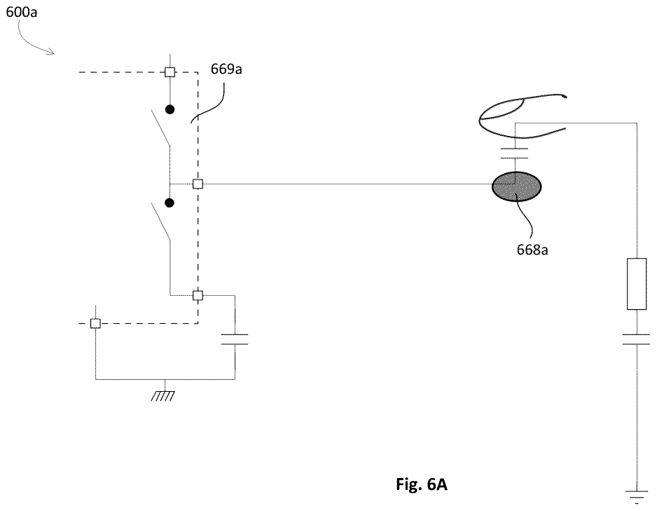

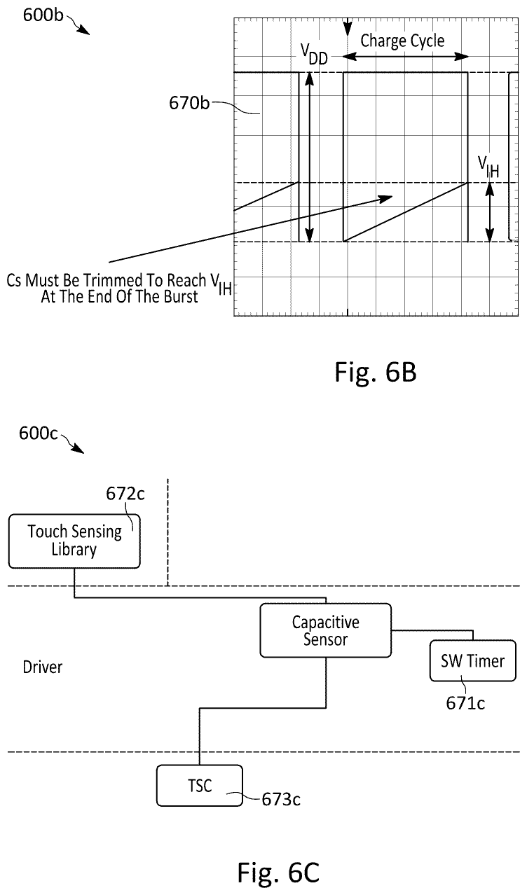

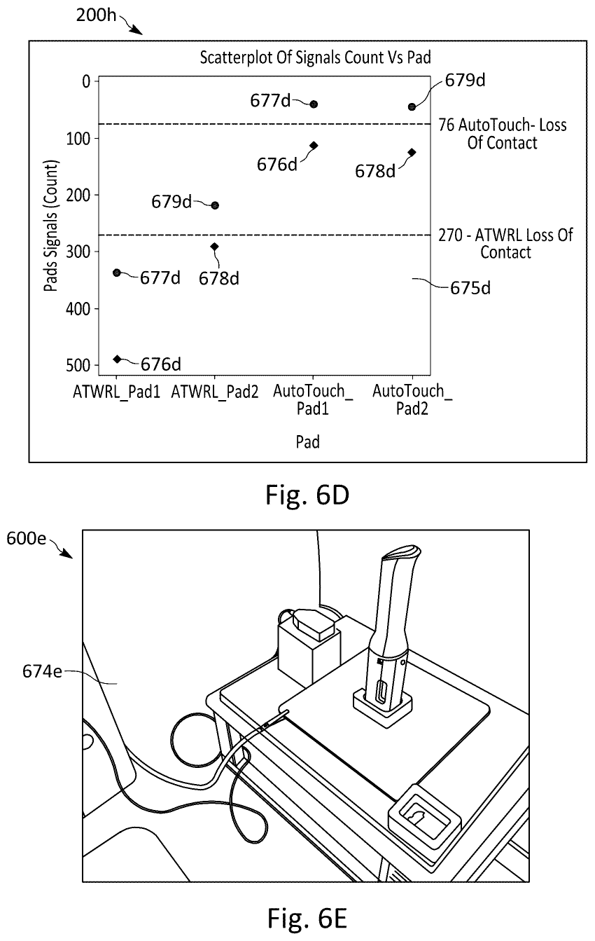

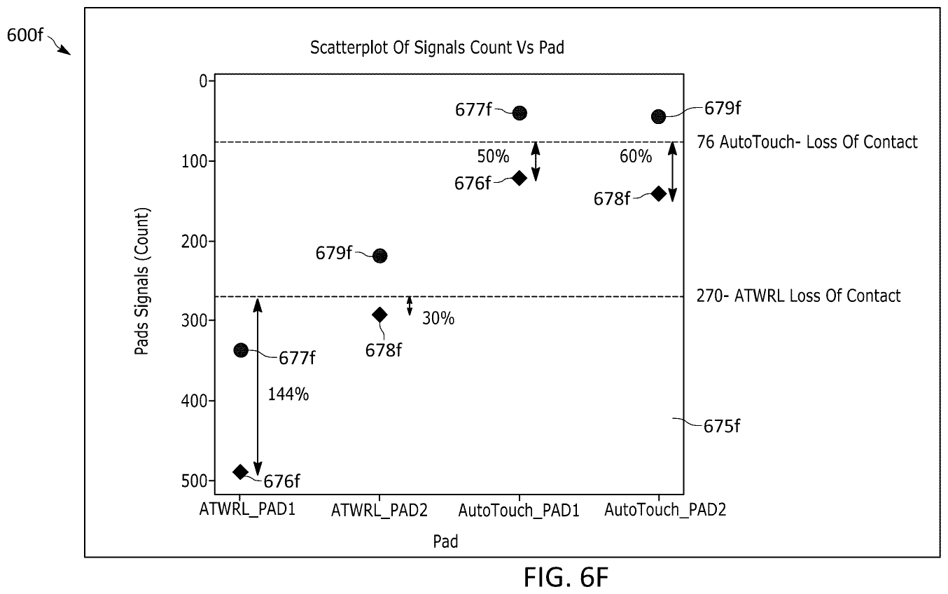

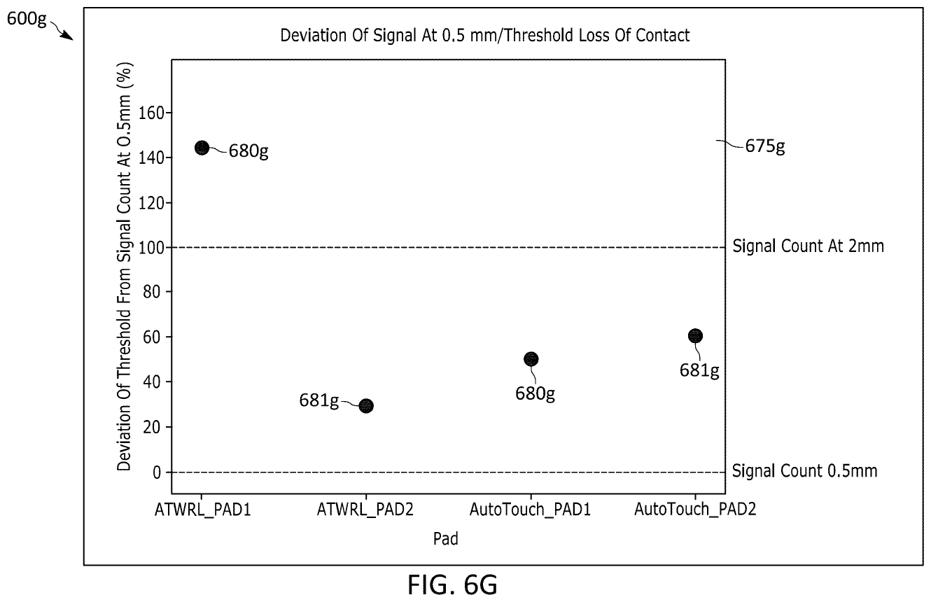

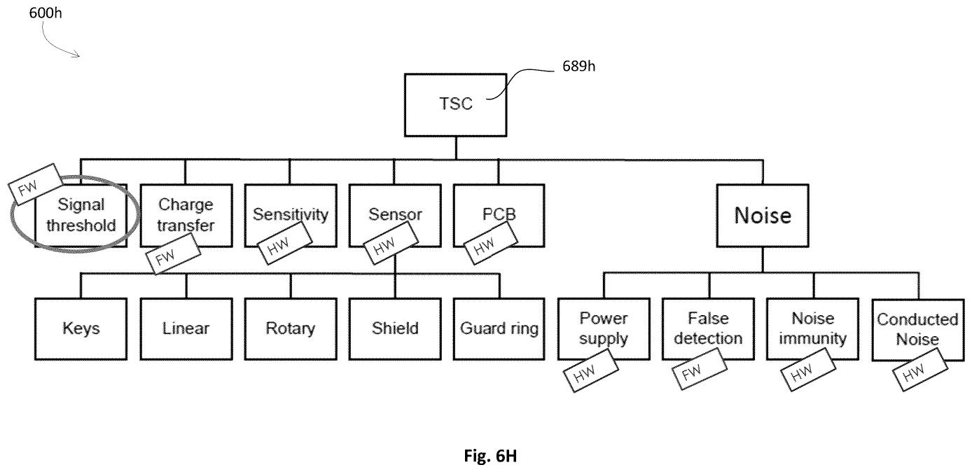

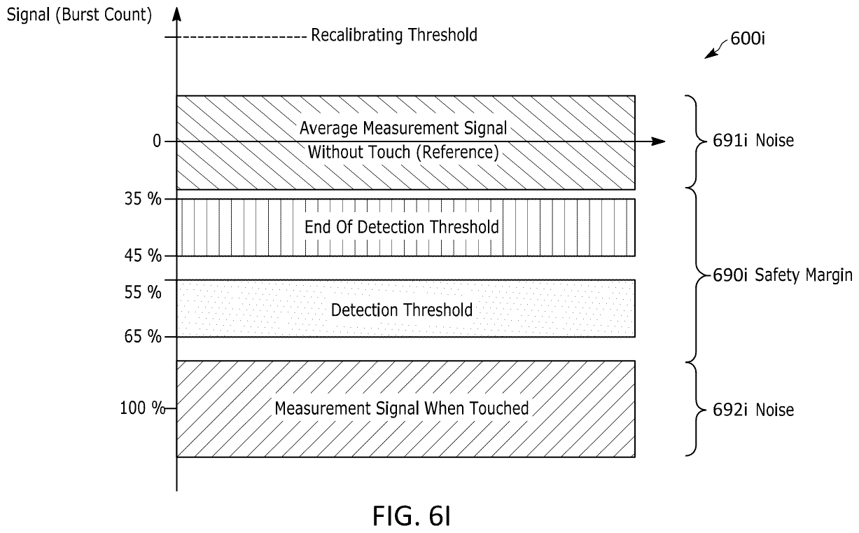

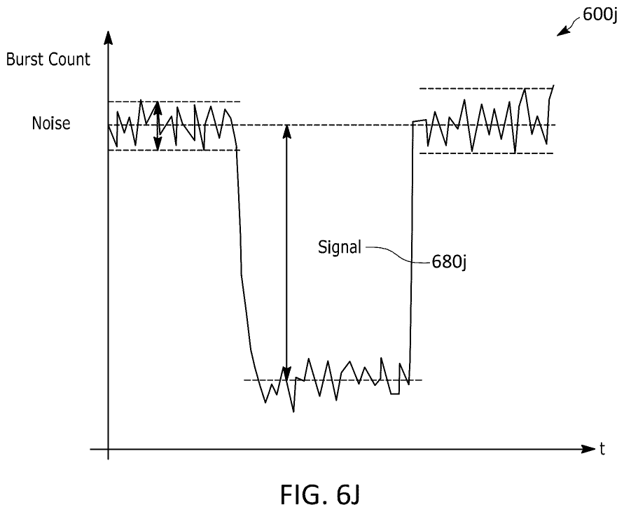

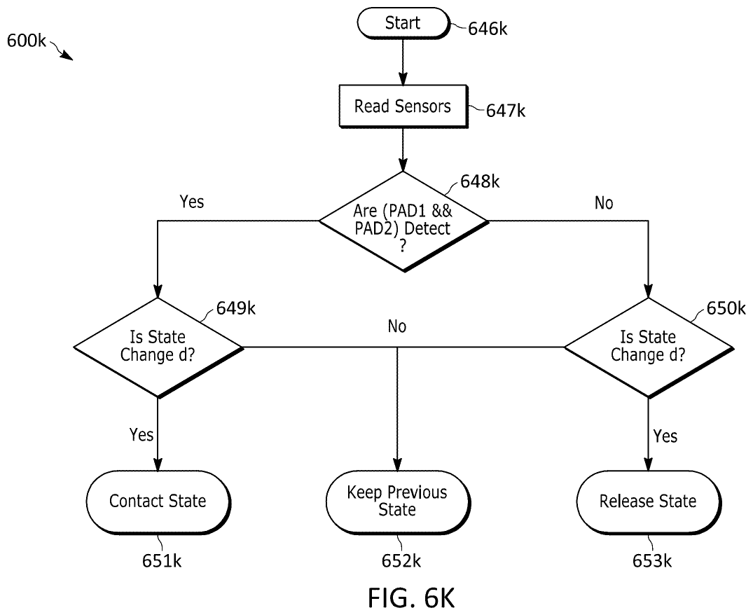

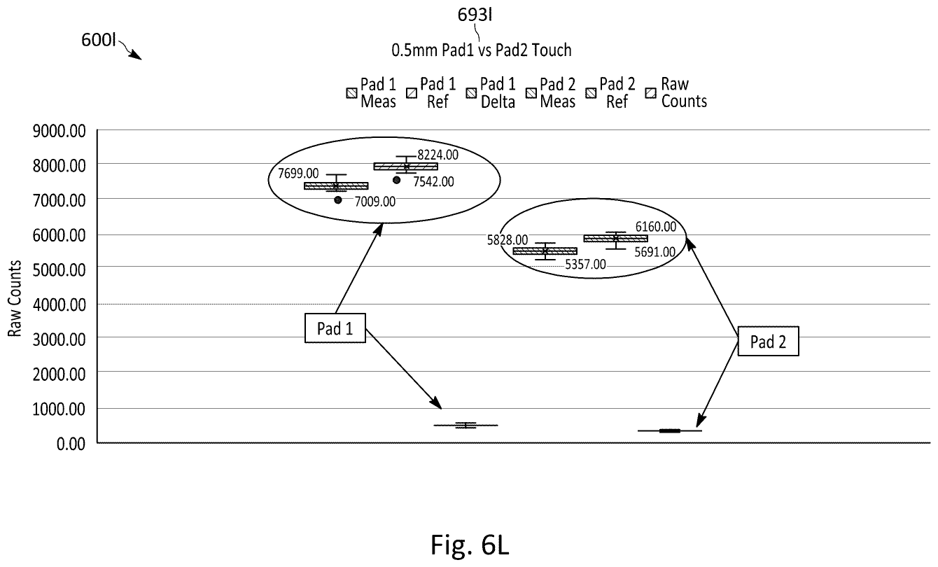

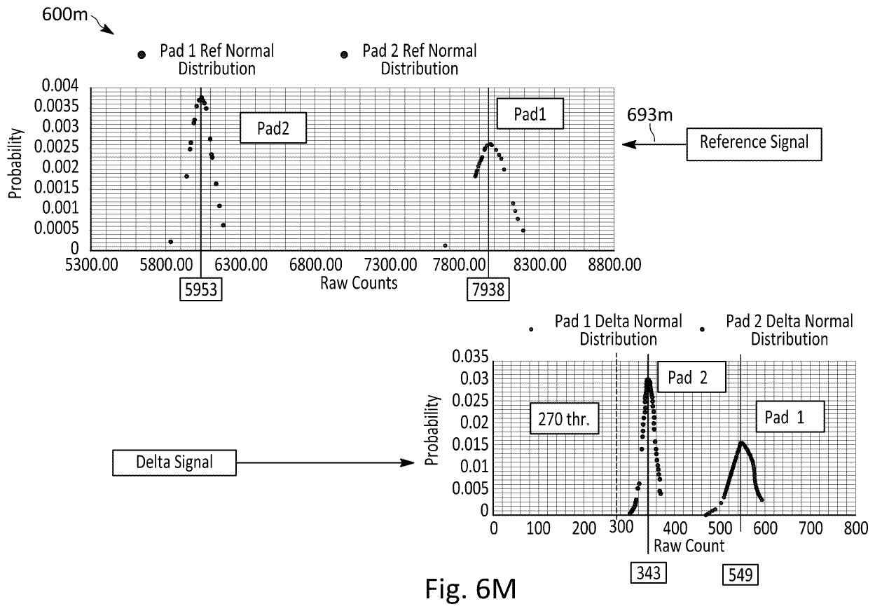

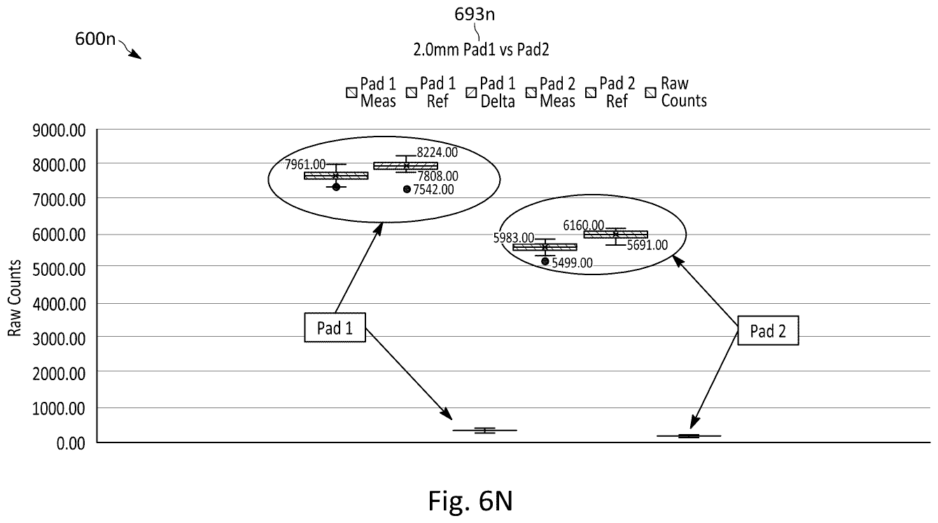

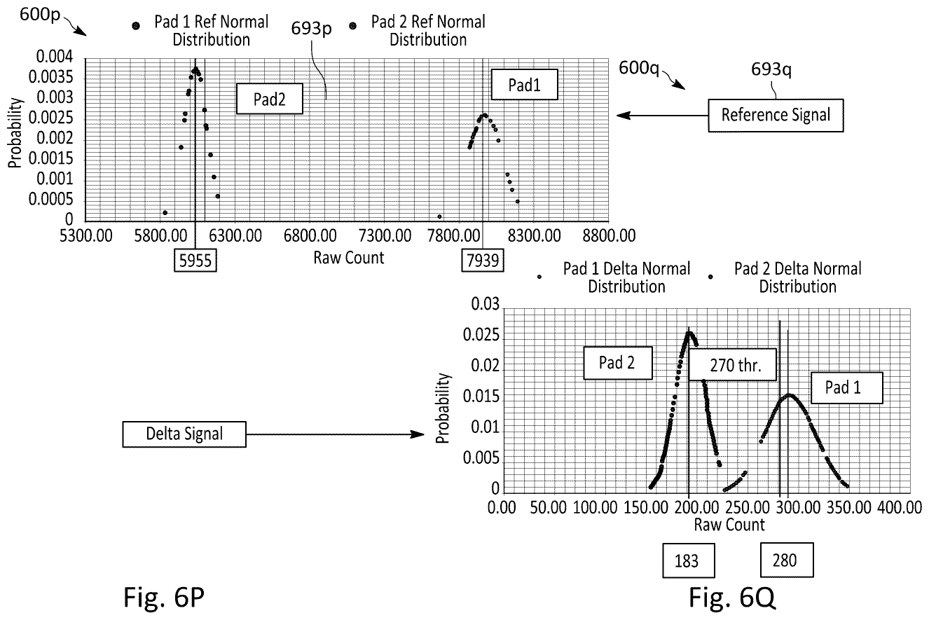

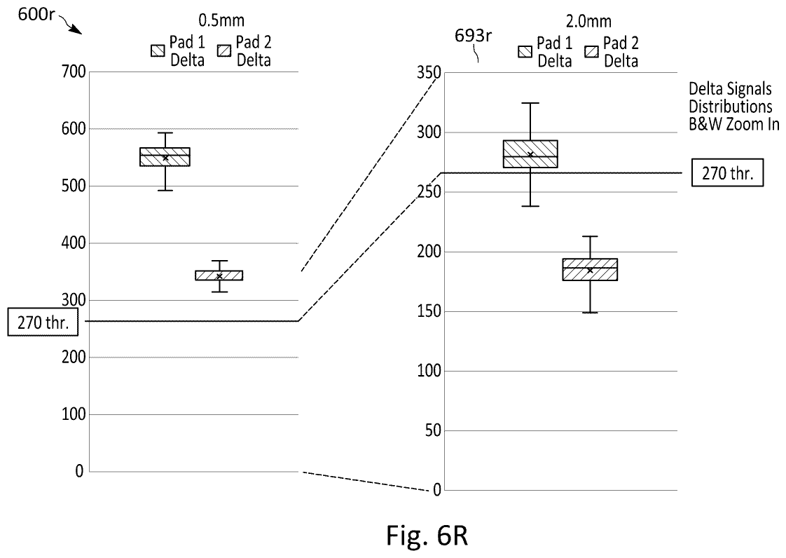

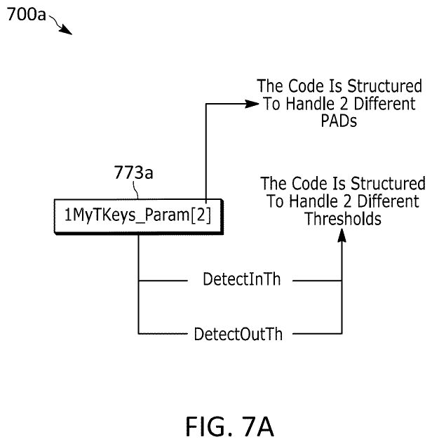

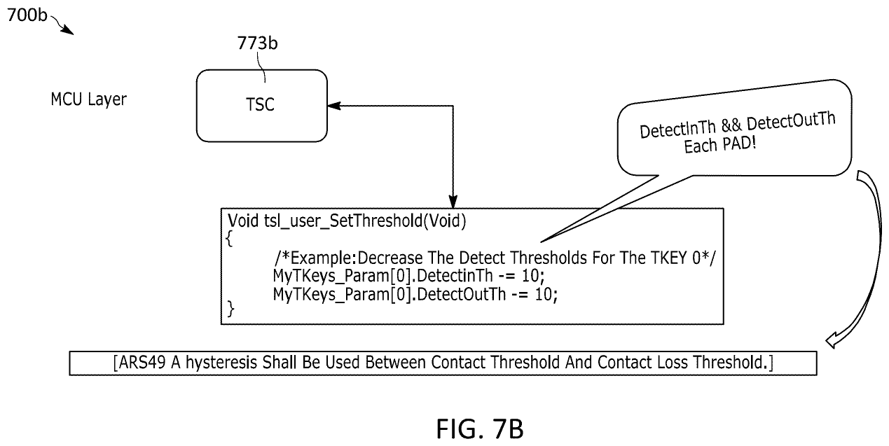

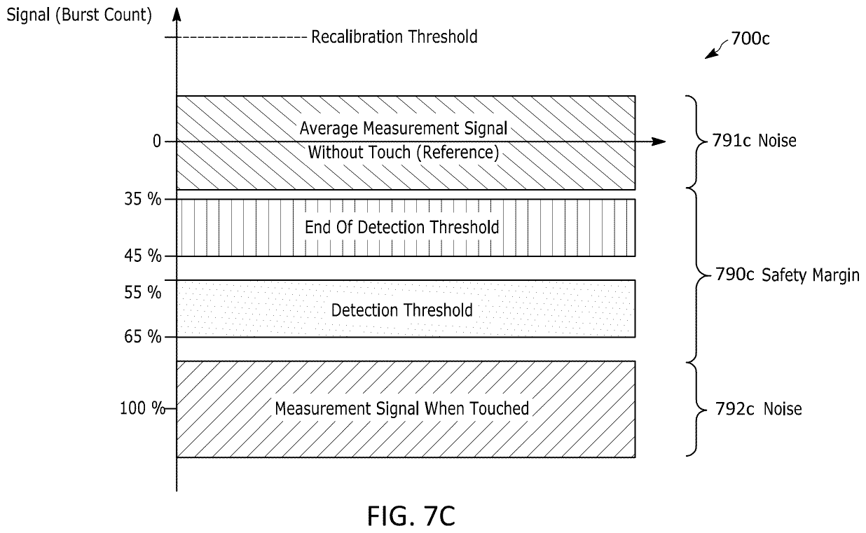

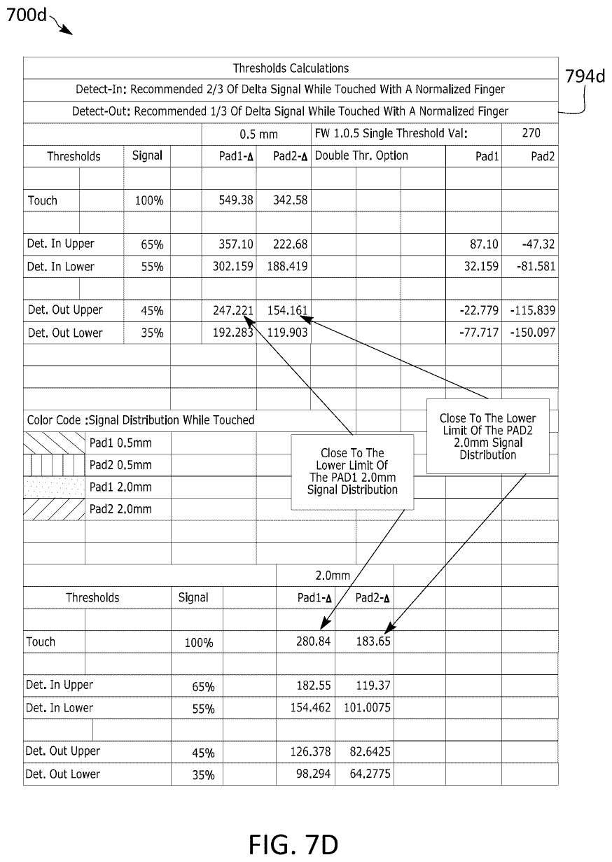

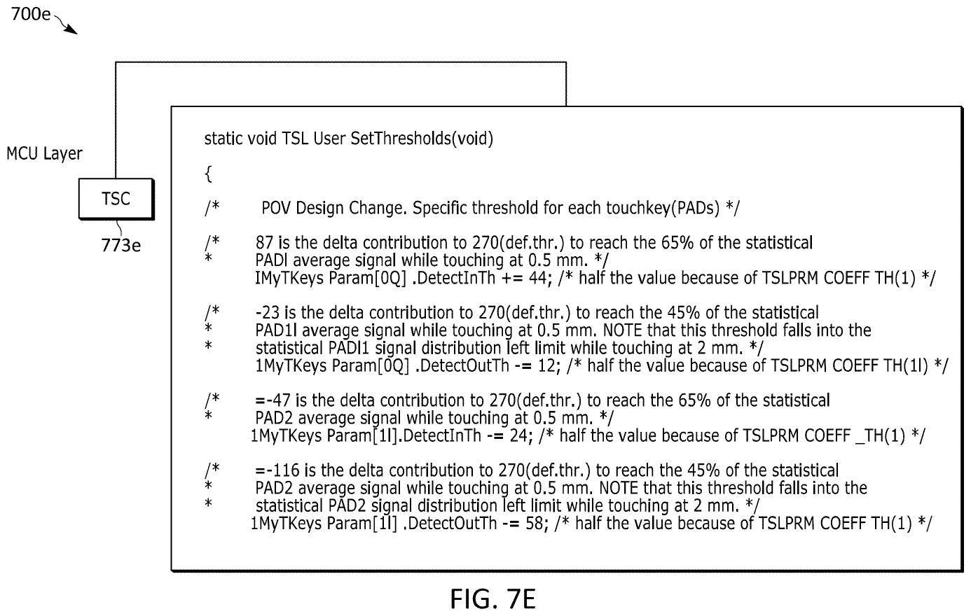

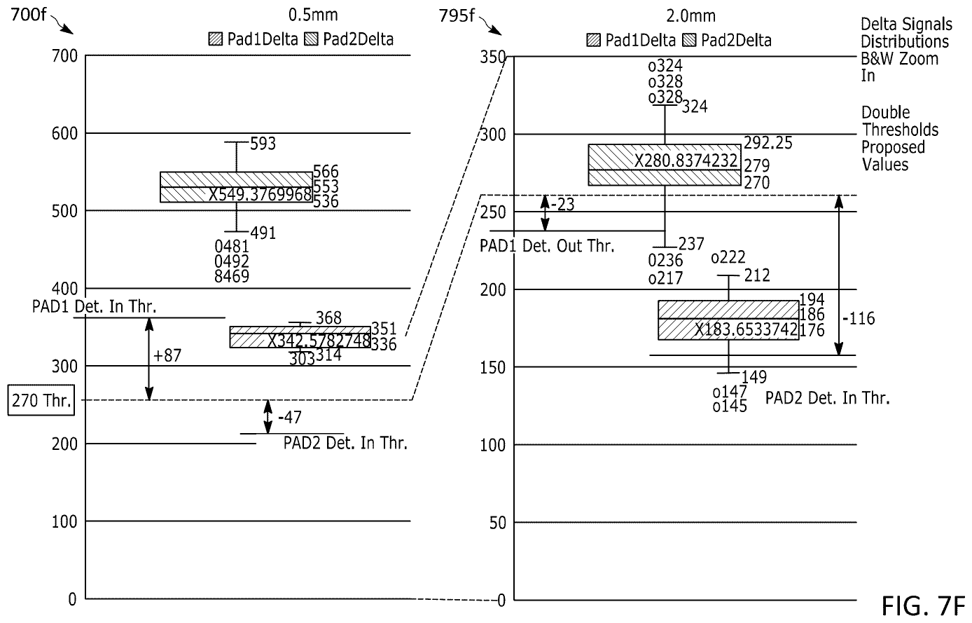

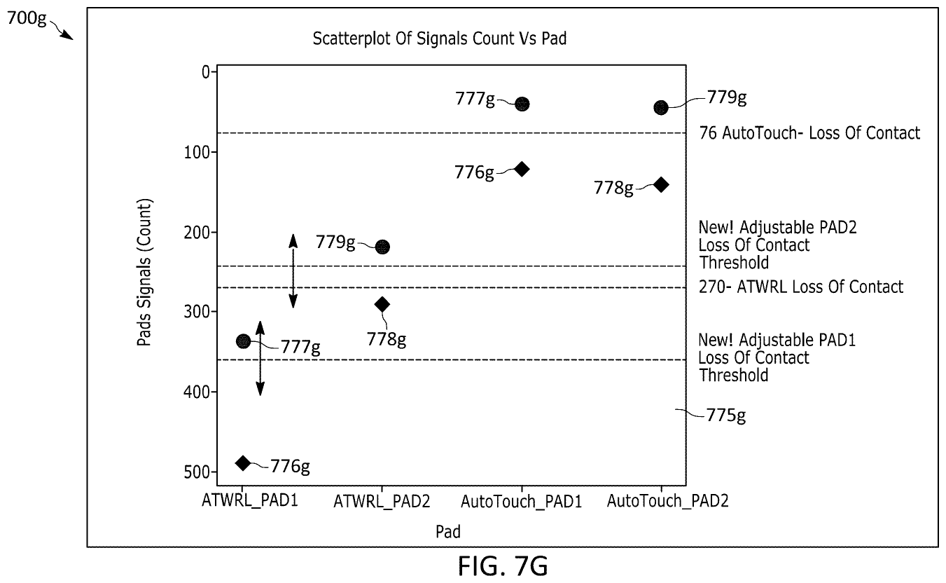

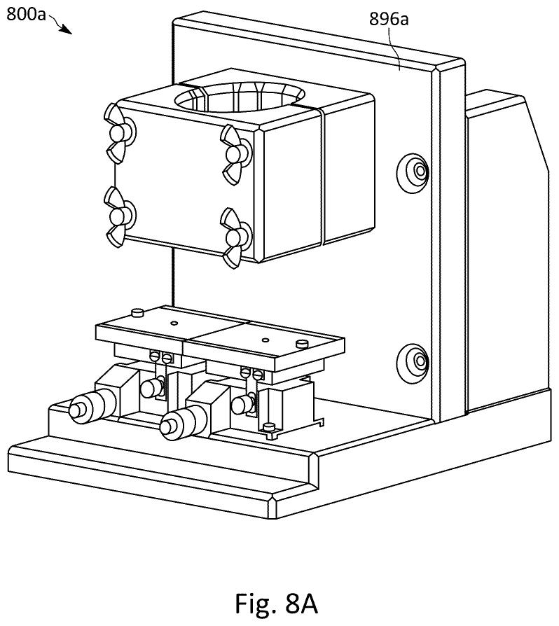

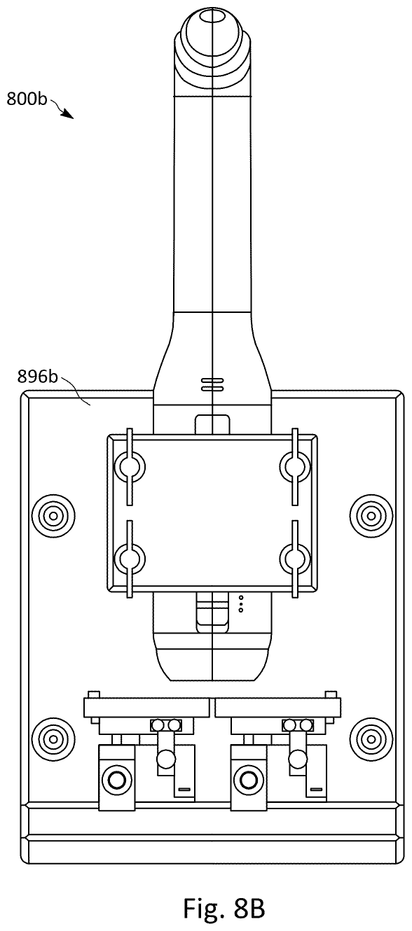

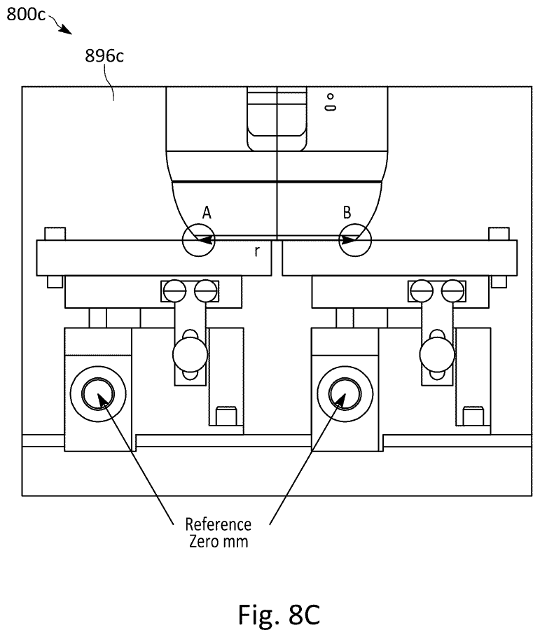

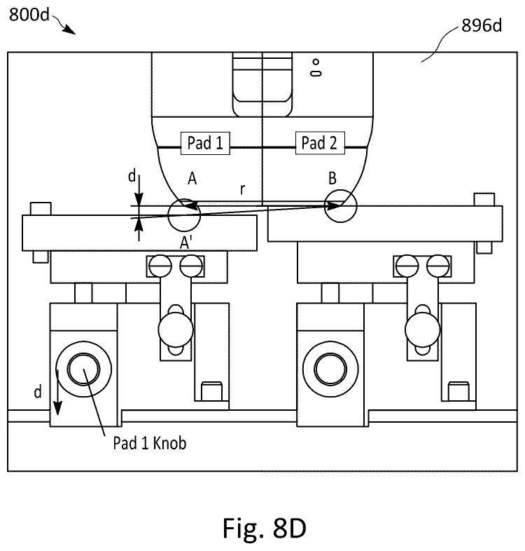

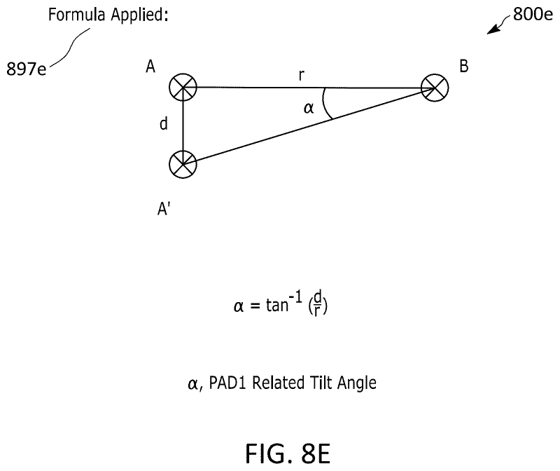

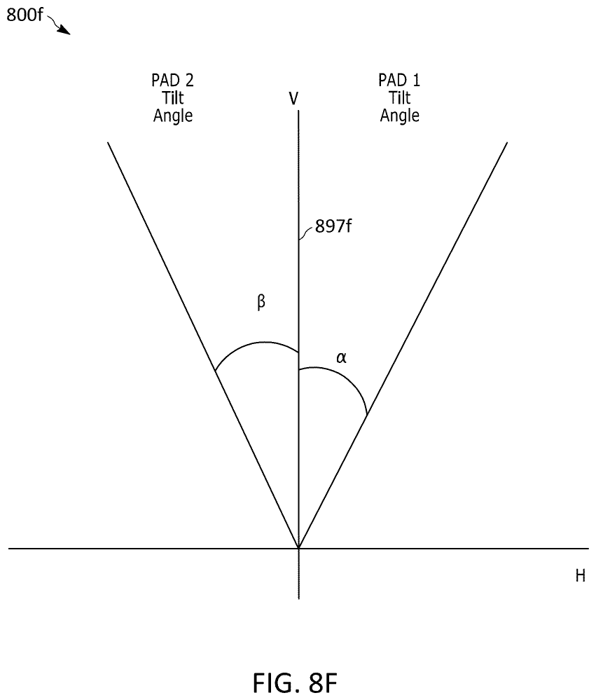

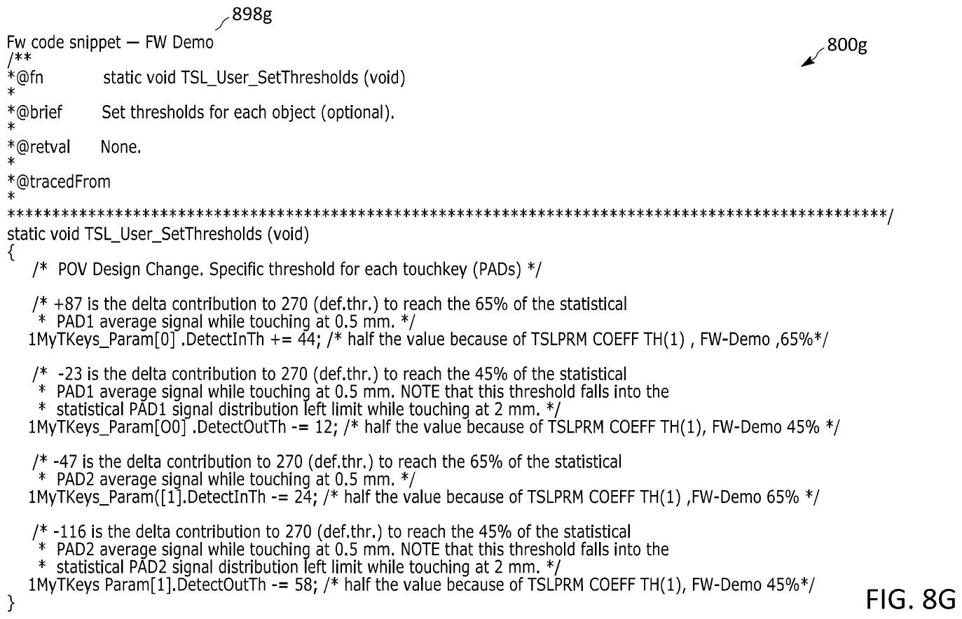

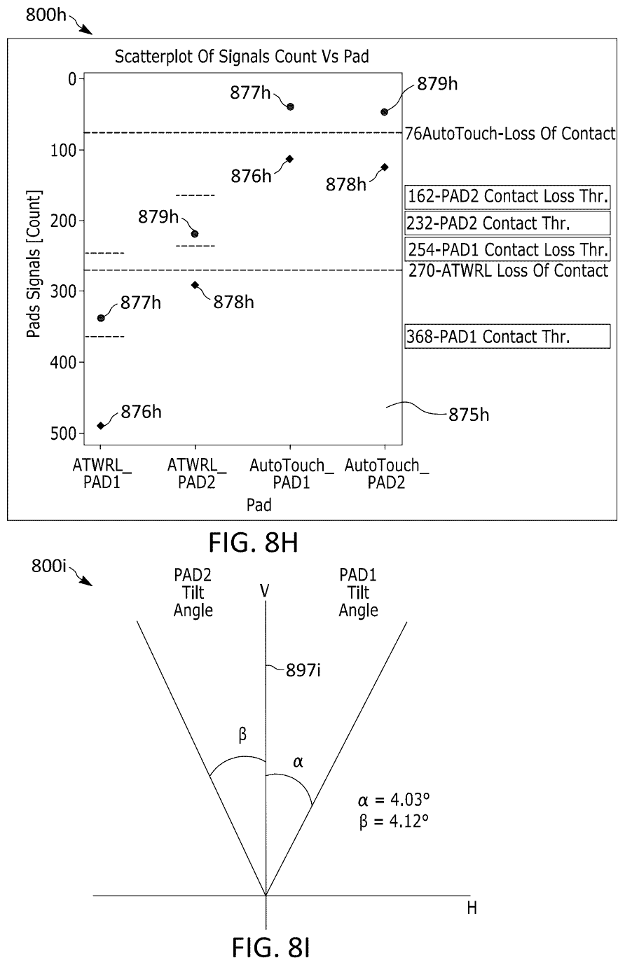

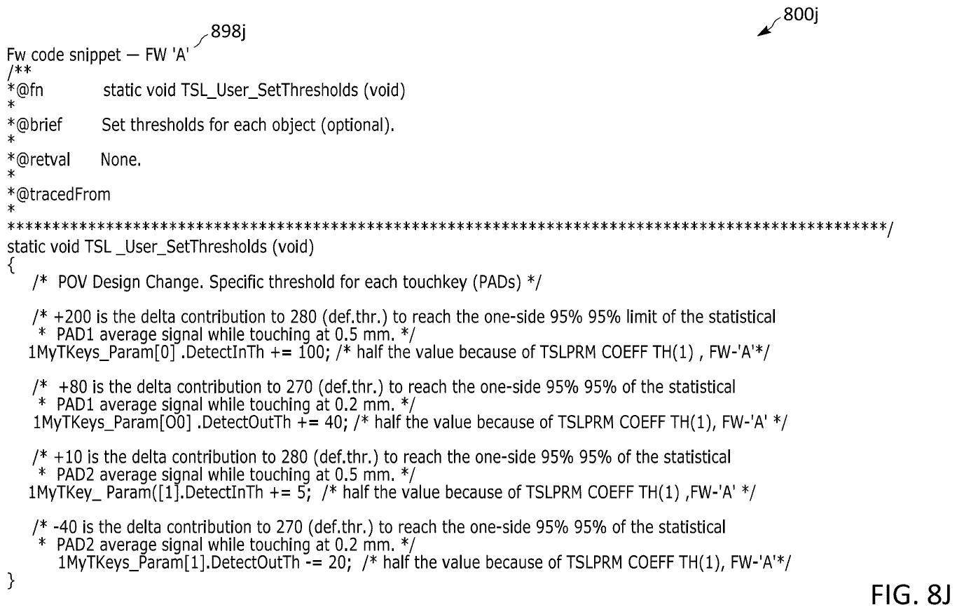

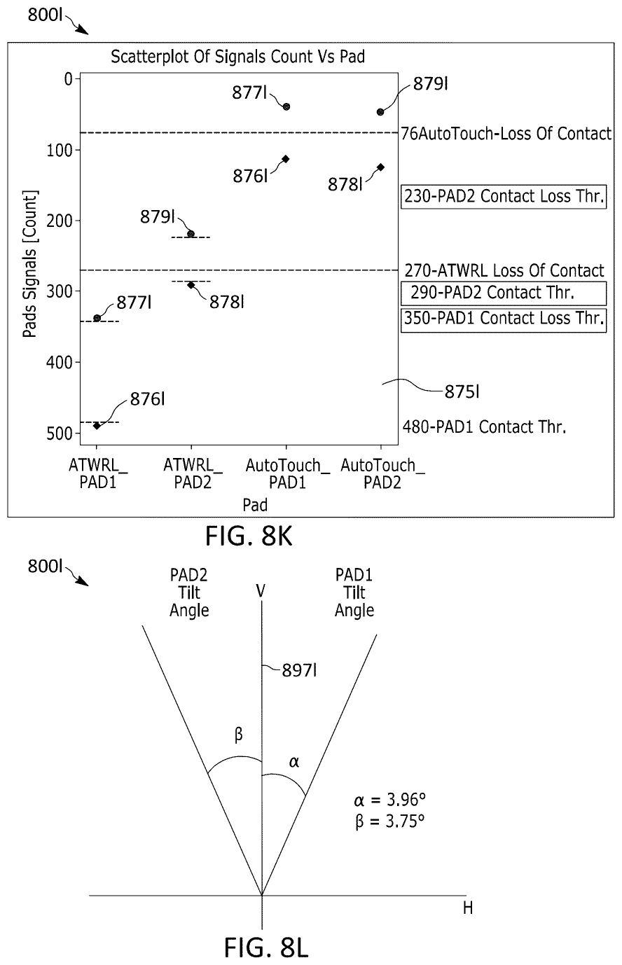

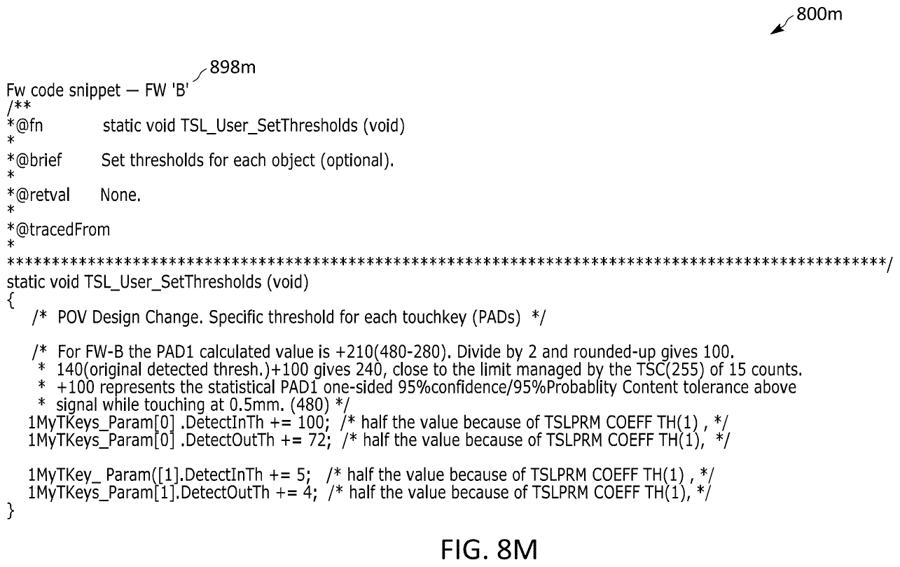

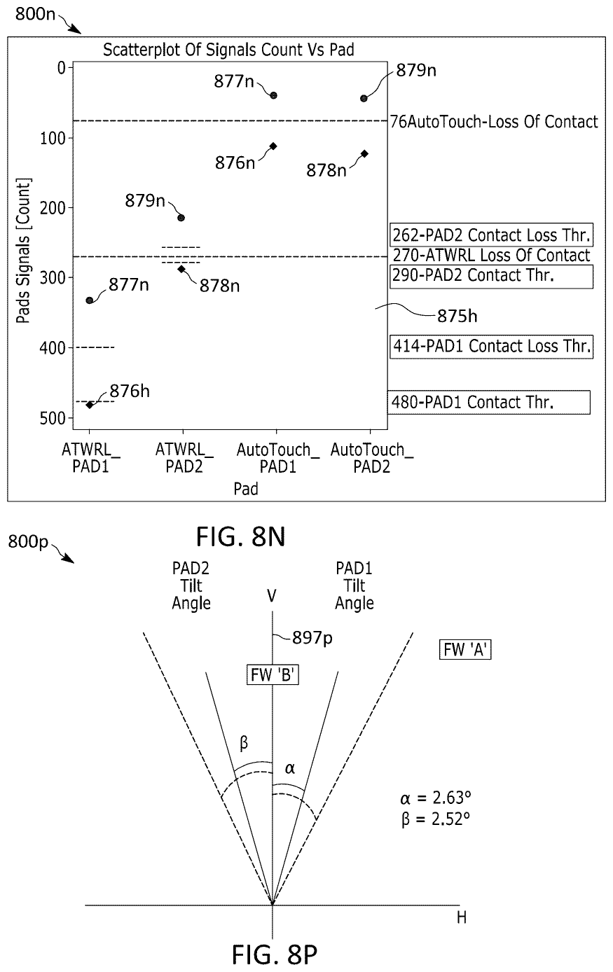

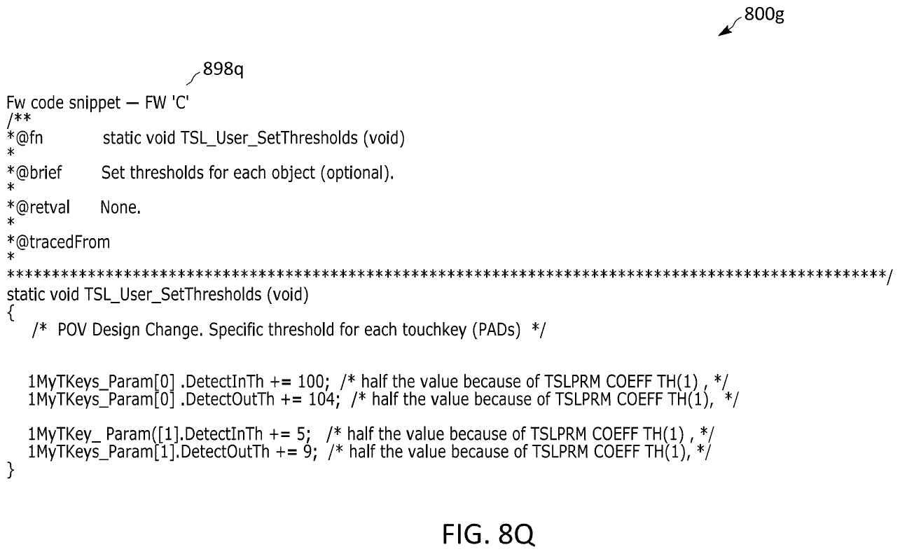

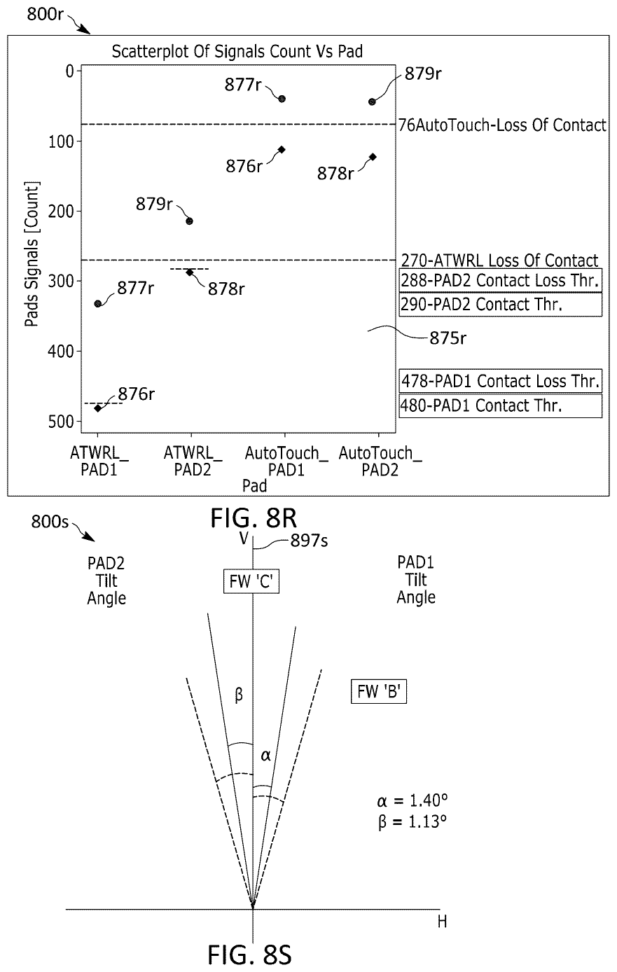

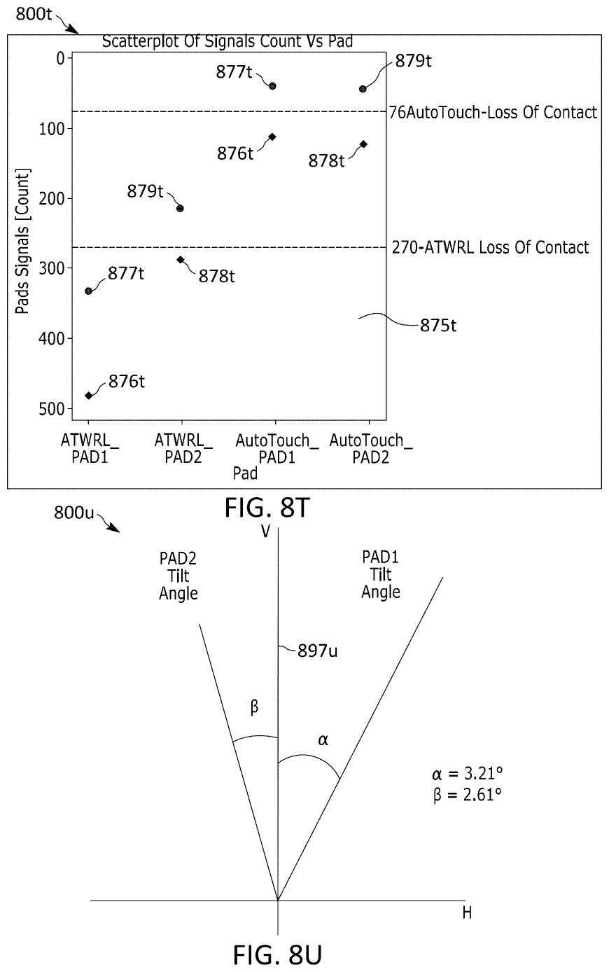

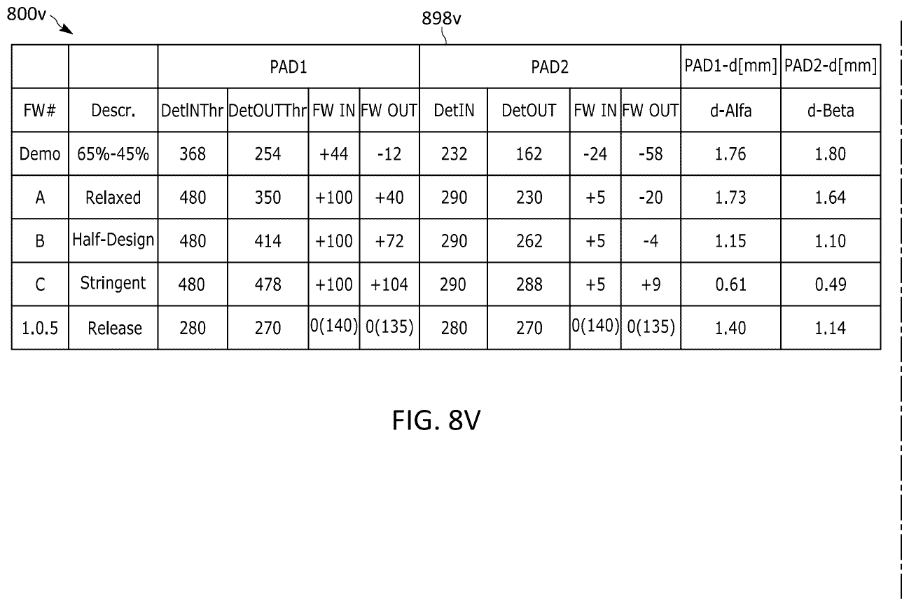

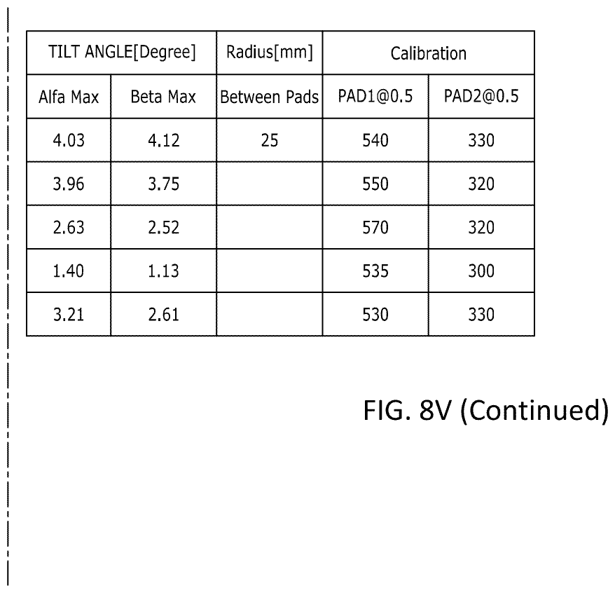

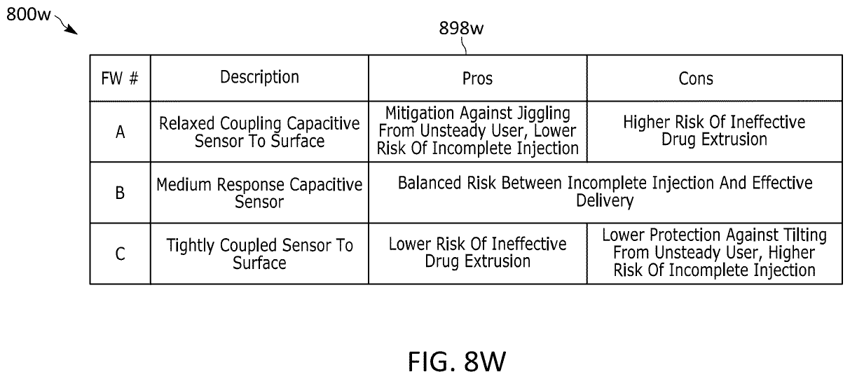

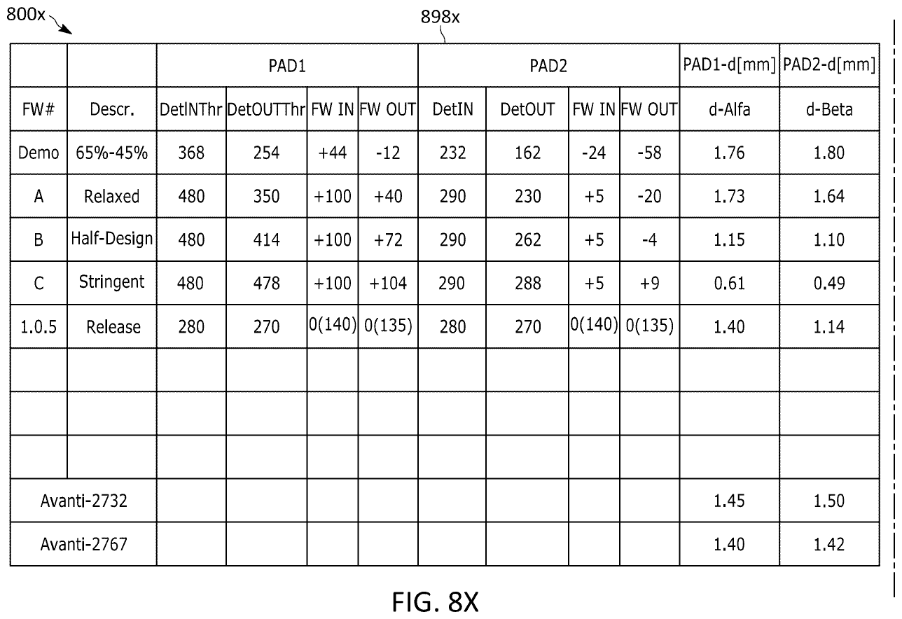

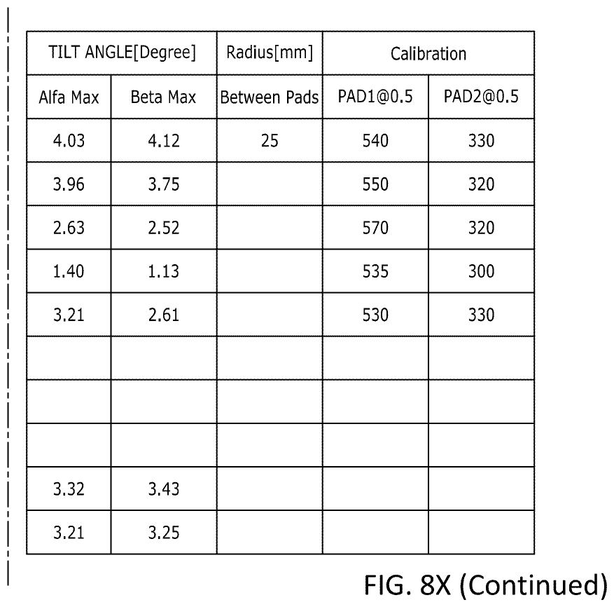

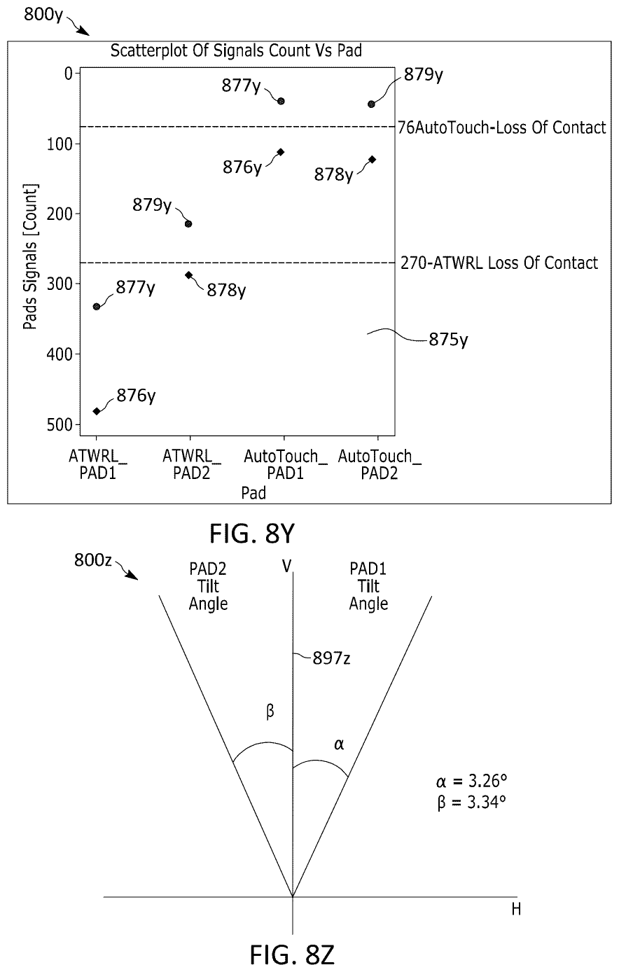

With reference to FIGS. 6A-N and P-R, a drug delivery device 600a-n,p-r may include two proximity sensors 234e, 235f. With addition reference to FIGS. 7A-G, a drug delivery device 700a-g may include thresholds for use with a proximity sensor. With further reference to FIGS. 8A-N and P-Z, a drug delivery device 700a-g may include thresholds for use with a proximity sensor. Capacitive sensors can be used in medical devices to detect presence of human skin based on difference in capacitance observed by the sensors. When detecting skin, it is important to enable flexibility in detection based on variances in normal use by the end user or manufacturing. A plurality of unique capacitive sensor thresholds may be developed through software to, for example, detect skin in an autoinjector build with STM32 microcontrollers or any chipsets utilizing a charge-transfer acquisition principle for detection of capacitive surfaces.

The change-transfer acquisition principle consists of charging a sensor capacitance and transferring the accumulated charge into a sampling capacitor, repeating until the voltage across the sampling capacitor reaches a max voltage. When the sensor detects a skin, capacitance to the earth is increased, thus the signal count and voltage required to reach the max voltage will decrease. When these values go below a defined threshold, software will indicate detection of skin.

Skin detection in an autoinjector requires software and hardware components. Hardware consists of microcontrollers utilizing a charge-transfer acquisition principle for detection of capacitive surfaces, including a touch sensing controller peripheral (TSC). Microcontroller software will be used to process signals and manage signal thresholds based on signals from the hardware, including a touch sensing library (TSL) API.

The TSL API allows for adjustment of each capacitive sensor channel independently. By adjusting individual parameters at the TSC level, unique thresholds can be applied by the TSL to each capacitive sensor channel independently. This allows for setting capacitive sensor thresholds to potentially compensate for variance in normal use by the end user, different skin types or conditions, or in manufacturing.

FIGS. 6A and 6B illustrate touch sensing most relevant acronyms are described below: Acquisition mode; CT: Charge-Transfer acquisition principle. This mode is used on STM32 microcontrollers; Touch sensing STM32 peripheral;—TSC: touch sensing controller peripheral; Sensors;—Touchkey or TKey: single channel sensor; STM32 software; TSL: touch sensing library; Delta: difference between the measure and the reference; Measure or meas: current signal measured on a channel; and Reference or ref: reference signal based on the average of a sample of measures.

The STM32 touch sensing feature is based on charge transfer. The surface charge transfer acquisition principle consists in charging a sensor capacitance (Cx) and in transferring the accumulated charge into a sampling capacitor (Cs). This sequence is repeated until the voltage across Cs reaches VIH. The number of charge transfers required to reach the threshold is a direct representation of the size of the electrode capacitance. When the sensor is touched, the sensor capacitance to the earth is increased. This mean the C voltage reaches VIH with less count and the measurement value decreases. When this measurement goes below a threshold, a detection is reported by the TSL Upper Statistical Limit thresholds 676_, 678_, 776_, 778_, 876_, and 878_, as used in FIGS. 6A-8Z, may be representative of, for example, a proximal end of a drug delivery device at 2 mm from an injection site. Lower Statistical limit thresholds 677_, 679_, 777_, 779_, 877_, and 879_, as used in FIGS. 6A-8Z, may be representative of, for example, a proximal end of a drug delivery device at 0.5 mm from an injection site.

With further reference to FIGS. 8A-N and P-Z, the FW change described introduced a new physical dimension to be evaluated: tilt angle. The evaluation cannot be performed with current DV test fixtures. A moving plates fixture was designed to further test the FW change. The provided fixture shall: purpose measure the distance when a PAD detects contact or looses contact from a moving (conductive) object. The provided fixture shall: requirements Enable the measure on each sensor PAD1 and PAD2 independently of one another. Provide a graduated scale in order to set the zero reference when the device capacitive sensor is touching the surface. Provide a handle or mechanism to move the conductive surface close or off of the device capacitive sensor. When the device is touching the entire capacitive sensor (display view) with both the surface the fixture reference shall be set to 0 mm. A: is the left geometrical reference point*; B: is the right geometrical reference point*; r: is the distance A-B**; (*) display view; and (**) equal to the bottom nose width.

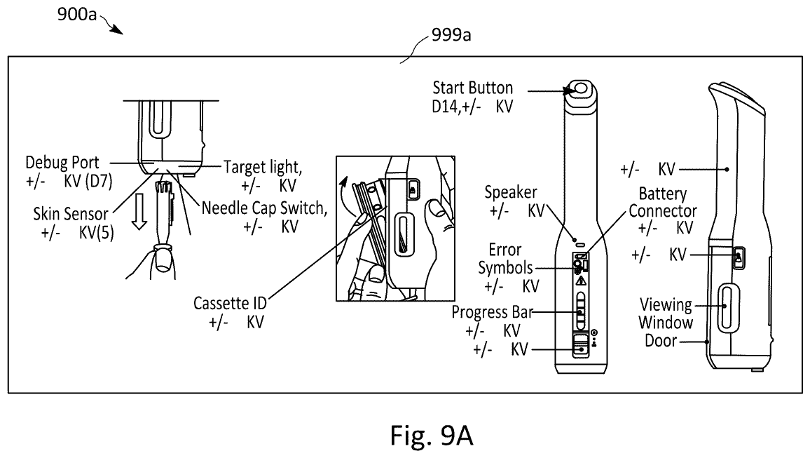

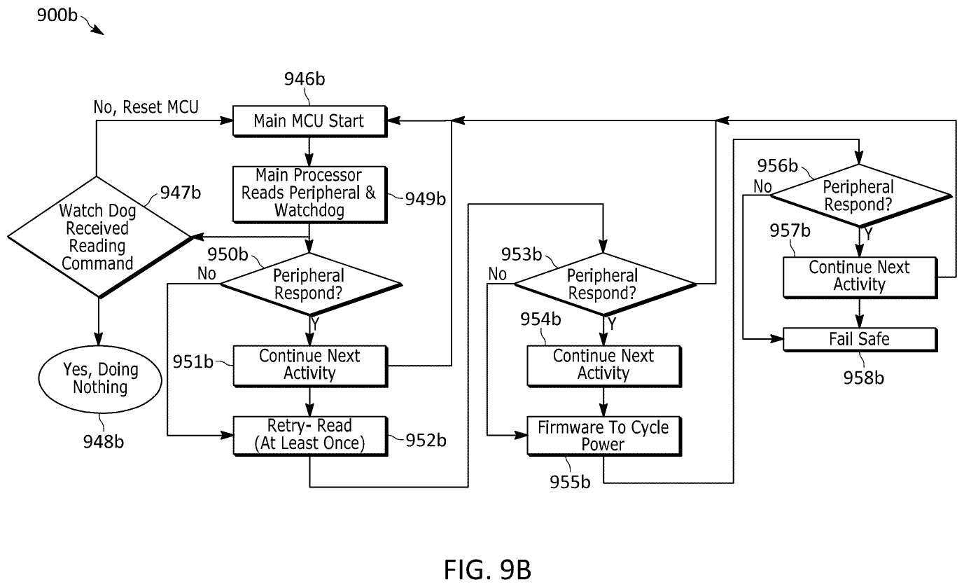

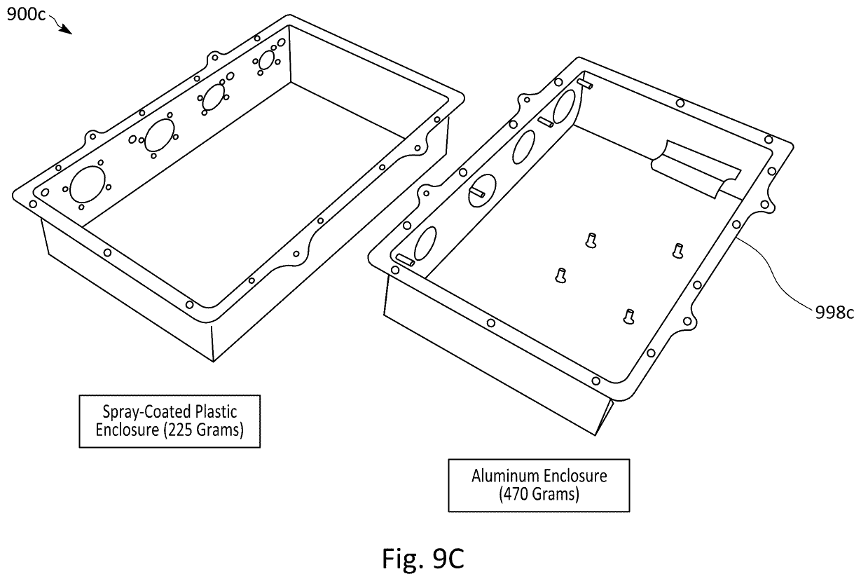

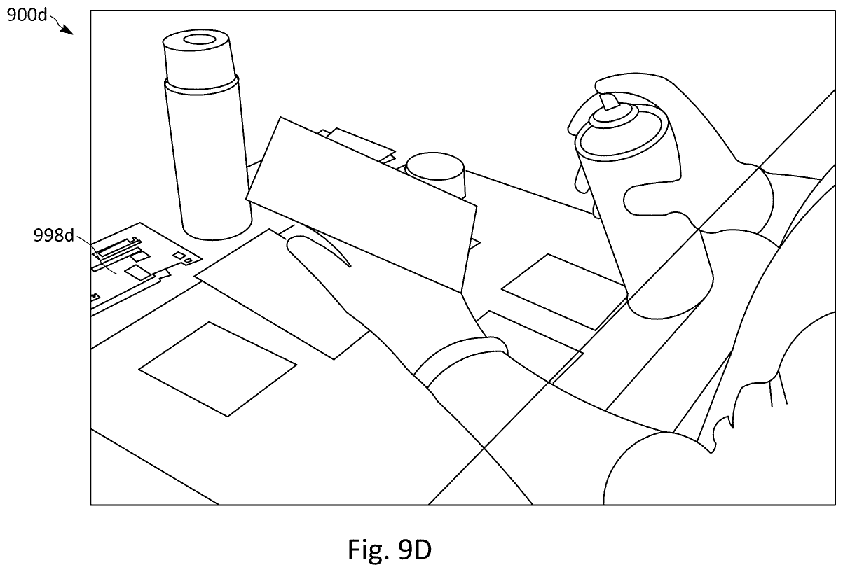

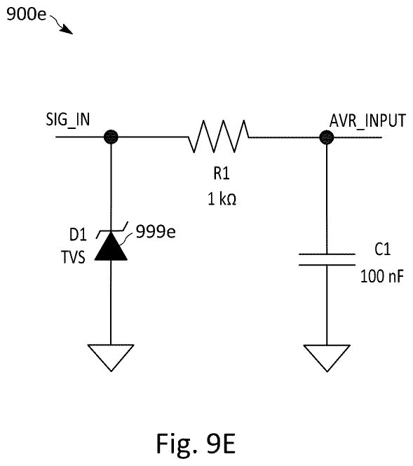

Turning to FIGS. 9A-E, electrostatic discharge (ESD) protection 900a-f may include hardware ESD 999e, 998d-f, firmware ESD protection 900b, and/or software ESD protection. The Electrostatic Discharge (ESD) have been a common factor causing portable or wearable electromechanical drug delivery device malfunction or damage that results in the missing dosage or delay therapy. ESD protection and recovery solutions may be accomplished through hardware architecture and firmware logics. This unique design considered the device formfactor and user interface that commonly make device vulnerable due to the ESD creeping for damaging or breaking semiconductor electronically inside. The design considered trade of cost, effectiveness and operational sequence in making the solution implementable and manufacturable for most of typical drug delivery devices.

Three factors contribute to ESD incident which are movement or friction, nonconductive material and dry air. The common static voltage can reach up to 30KV in the dry environment which can easily interferences electronic stability and life. Portable or wearable drug delivery device such as autoinjector, mini infuser, patch pump or on body injector are commonly used in the environment with the three factors above and therefore interference or damaged by ESD. Since drug delivery devices consist of injection site detection portion which contacts patient skin or body, needle insertion interface which connects drug fluid to patient tissue, hand holding area and activation button which contacts patient hand, and debug port connects to test equipment, all these ports need to have ESD protection. These ports lead ESD current into device enclosure. Duration operation, different area of device contacts patients at different time sequence, duration or different press pressure that will need different protection level accordingly, for example, usually patient loads cassette first by pressing cassette door ejection button and then closes door afterwards and then put device on injection site. Since cassette door ejection button, injection site contact will contact patient first, they need higher ESD protection voltage. On the other hand, some areas that don't contact patient first don't have to have that high ESD voltage protection to save material cost. Moreover, based the patient contact distance to the internal ESD sensitive components, the autoinjector may be equipped with different level of ESD protection with different ESD protection circuit or component for this purpose as well. Furthermore, for some non ESD sensitive area, ESD protection is not necessary. This disclosure documents the unique design from hardware and firmware design respectively to implement the different ESD voltage protection level to mitigate damage effectively and economically. The FIG. 9A illustrates the hardware protection solution based on the ESD voltage analysis and user contact sequence and frequency. In general, user interface for device operation such as cassette door ejection button for loading/unloading has higher ESD voltage, activation button has lower ESD voltage which implemented with different ESD suppressors to reduce bill of material or (manufacture of goods) cost.

Since many times ESD doesn't permanently damage electronic components instead it puts semiconductor in a wrong state (binary 0/1 reversed state) and then caused device malfunction or freeze. When this situation happened, the drug delivery device designed a logical to recover the malfunctions. When user interface portion of circuit is in the malfunction state or freezing mode, main processor will attempt to reset or power cycle the portion of circuit to recover it. If the circuit is recovered, the device resumes the left over procedures. Since an automatic recovery process may, for example, only take milliseconds, user will not notice the recovering process under hood which gives patients confidence for the treatment. If the portion of circuit is not recoverable, the event will be logged for debugging and analysis purpose and then fail gracefully. Firmware recovery doesn't add cost to the bill of material. If main processor was interrupted or damaged by ESD, watch dog circuit will kick in to reset the entire system to attempt to recover which is the same logic as main processor recover user interface related circuits.

FIG. 9B demonstrates the firmware logic for recovering the ESD interference event. The main microprocessor may start (block 946b) and a watchdog circuit input may be received (block 947b). If no watchdog reading commands are received in block 947b, an OK watchdog timer reset may be generated (block 948b). A watchdog signal (e.g., a status in memory, a status register, etc.) may be read by the main processor (block 949b). A determination regarding the watchdog signal is made (block 950b). If the peripheral indicates a response block 950b, the process may continue (block 951b). If the peripheral indicates no response block 950b, at least one peripheral read retry may be attempted (block 952b). As illustrated in FIG. 9B, the “retry” may be attempted at least one time (blocks 950b-958b) before a drug delivery device ESD error may be determined.

As a specific example, when a main processor reads a watch dog circuit status, the watch dog may know the read occurred and then may reset watch dog timer without triggering resetting a main processor signal. The main processor may, for example, read a watch dog and indicate to the watch dog that the watch dog was read by the main processor (i.e., not meant to read data per se). A watch dog may include a timer. If the watch dog is not read by the main processor in a pre-set time, the watch dog may send a signal to reset the main processor (i.e., if the watch dog is not read by the main processor within a preset time period that means main processor may be frozen, logic may include an error, etc.). A watch dog circuit may be configured as a very simple component. The watch dog circuit may resist ESD and may not easily be damaged, inadvertently interrupted, etc.

A mechanical ESD protection/recovery solution may include, for example: make device conductive; or design Insulation/distance between enclosure and electronic component. An electronic solution may include, for example, adding ESD protective component/circuit on hardware. Software ESD protection/recovery solutions may include, for example, mask ESD errors by adding watchdog circuit and software recovery.

The above description describes various devices, assemblies, components, subsystems and methods for use related to a drug delivery device such as a pre-filled syringe. The devices, assemblies, components, subsystems, methods or drug delivery devices (i.e., prefilled syringe) can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir within the pre-filled syringe for example. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF: cMet axis (HGF/SF: c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris ™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL 12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF α monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 350) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BITER (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vill (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein. Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device, comprising:
a housing configured to carry a syringe with a medication;
an extrusion drive for selectively extruding the medication from the syringe during an injection process; and
a main microcontroller and a wireless communication module carried by the housing;
wherein the main microcontroller and the wireless communication module are communicatively connected via a communication channel,
wherein the main microcontroller includes a first real-time clock,
wherein the main microcontroller is configured to generate injection data based on the first real-time clock,
wherein the wireless communication module includes a periphery interface and a second real-time clock, and
wherein the periphery interface is configured to communicate the injection data to a remote device based on the second real-time clock.

2. The drug delivery device of claim 1, wherein the main microcontroller is configured to automatically control at least a portion of a medication injection process.

3. The drug delivery device of claim 1, further comprising:
an external wireless device connected and paired to the periphery interface, wherein the second real-time clock is for synchronization.

4. The drug delivery device of claim 1, further comprising:
a memory, wherein the main microcontroller is configured to automatically store the injection data in the memory.

5. The drug delivery device of claim 4, wherein the wireless communication module is configured to read injection data from the memory.

6. A method of operating a drug delivery device, the method comprising:
providing a main microcontroller communicatively connected with a wireless communication module via a communication channel,
wherein the main microcontroller includes a first real-time clock,
wherein the main microcontroller is configured to generate injection data based on the first real-time clock and to control at least a portion of a medication injection process based on drug delivery device configuration data,
wherein the wireless communication module includes a periphery interface and a second real-time clock; and
communicating injection data, via the periphery interface, based on the second real-time clock.

7. A method as in claim 6, wherein the main microcontroller is configured to automatically control at least a portion of a medication injection process.

8. A method as in claim 6, wherein the wireless communication module is configured to transmit injection data to an external wireless device via the periphery interface.

9. A method as in claim 6, wherein the wireless communication module includes a Bluetooth low energy (BLE) device.

10. A method as in claim 6, wherein external wireless device is Bluetooth low energy (BLE) capable.

11. A method as in claim 6, wherein a time-stamp is based on the first real-time clock when the main microcontroller is active.

12. A non-transitory computer-readable medium storing computer-readable instructions that, when executed by one or more processors, causes the one or more processors to:
receive a first real-time clock signal from a main microcontroller communicatively connected with a wireless communication module via a communication channel, wherein the main microcontroller is configured to generate injection data and to control at least a portion of a medication injection process;
receive a second real-time clock signal from the wireless communication module; and
communicating the injection data via a periphery interface of the wireless communication module based on the second real-time clock.

13. The non-transitory computer-readable medium of claim 12, wherein the main microcontroller is configured to automatically control at least a portion of a medication injection process.

14. The non-transitory computer-readable medium of claim 12, further comprising:
connecting and paring an external wireless device to the periphery interface, wherein the second real-time used clock is for synchronization.

15. The non-transitory computer-readable medium of claim 12, further comprising:
automatically storing injection data in a memory using the main microcontroller.

16. The non-transitory computer-readable medium of claim 12, wherein the wireless communication module is configured to retrieve injection data from the main microcontroller.

17. The non-transitory computer-readable medium of claim 12, wherein at least one of: a time read request or a date read request is based on the second real-time clock when the main microcontroller is in a sleep mode.

18. The non-transitory computer-readable medium of claim 12, wherein the communication channel is selected from a group including: a UART channel, an I2C channel, a SPI channel, or a GPIO channel.

19. The non-transitory computer-readable medium of claim 12, wherein the first real-time clock is used for keeping track of date and time-stamps correlated with injection data.

20. The non-transitory computer-readable medium of claim 12, wherein the drug delivery device is configured to deliver a medication based on the first real-time clock.

21-100. (canceled)
